(19) United States
(12) Patent Application Publication
(10) Pub. No.: US 2004/0091945 A1
(43) Pub. Date: May 13, 2004

(54) Peptides and methods of screening immunogenic peptide vaccines against Alzheimer's Disease

(76) Inventors: Cheryl Fitzer-Attas, Rehovot (IL); Daniel G. Chain, Jerusalem (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/619,454

(22) Filed: Jul. 16, 2003

Related U.S. Application Data
(60) Provisional application No. 60/396,245, filed on Jul. 17, 2002.

(51) Int. Cl. G01N 33/53; G01N 33/567; C07K 14/47

(52) U.S. Cl. 435/7.2; 530/350

(57) ABSTRACT

The invention is in the field of immunogenicity. In one embodiment, the invention relates to method of identifying T-cell epitopes in amyloid beta peptide or homologue thereof. In another embodiment, the invention relates to a vaccine comprising an amyloid beta peptide or homologue thereof, whereby the selected peptide is a peptide which lacks certain T-cell epitopes or a peptide which is modified by deleting or modifying amino acids so as to reduce or eliminate the T-cell epitopes. The selected peptides are further assessed for reduced capacity to form fibrils, reduced cytotoxicity, and a reduced ability to induce a cellular autoimmune response. The selected peptides are further assessed for ability to induce a humoral immune response. In another embodiment, the invention relates to a method of predicting the reaction of an individual to a vaccine, which comprises amyloid beta peptide or homologue thereof, based on the HLA haplotype of the subject. In another embodiment, the invention provides a method for matching a vaccine comprising amyloid beta peptide or homologue thereof to an individual, based on the HLA haplotype of that individual. In another embodiment, the invention provides a vaccine comprising an amyloid beta peptide or homologue thereof, whereby the amyloid beta peptide or homologue thereof, lacks the ability to induce a T-cell response

### CROSS REFERENCE TO RELATED TO APPLICATIONS

This application claims the priority of U.S. Provisional Application Serial No. 60/396,245, filed Jul. 17, 2002, which is incorporated in its entirely by reference herein.

### FIELD OF THE INVENTION

This invention is directed to peptides and methods of screening immunogenic peptides against Alzheimer's Disease. The invention relates to a method of identifying T-cell epitopes in amyloid beta peptide or homologue thereof. The invention also relates to amyloid beta peptide or homologue thereof and vaccine comprising an amyloid beta peptide or homologue thereof, whereby the amyloid beta peptide or homologue thereof are selected according to their lack of harmful T-cell epitope or are modified by deleting or modifying amino acids so as to reduce the T-cell epitopes. The invention further relates to a method of predicting the reaction of an individual to a vaccine, which comprises an amyloid beta peptide or homologue thereof, based on the HLA haplotype of the subject. In addition, the invention provides a method for matching a vaccine comprising amyloid beta peptide or homologue thereof based on the HLA haplotype of the individual.

### BACKGROUND OF THE INVENTION

A major histopathological hallmark of Alzheimer's Disease (AD) is the presence of amyloid deposits within neuritic and diffuse plaques in the parenchyma of the amygdala, hippocampus and neocortex (Glenner and Wong, 1984; Masters et al., 1985). Amyloid is a generic term that describes fibrillar aggregates that have a common P -pleated structure. These aggregates exhibit birefringent properties in the presence of Congo red and polarized light (Glenner and Wong, 1984). The diffuse plaque is thought to be relatively benign in contrast to the neuritic plaque which appears to be strongly correlated with reactive and degenerative processes. One of the principal components of neuritic plaques is a 42 amino acid residue amyloid-β (Aβ) peptide (Roher et al., 1993) that is derived from the much larger b amyloid precursor protein, β APP (or APP). A β 1-42 is produced less abundantly than the 1-40 A β peptide (Haass et al., 1992; Seubert et al., 1992), but the preferential deposition of A β 1-42 results from the fact that this COOH-extended form is more insoluble than 1-40 A β and is more prone to aggregate and form anti-parallel β-pleated sheets. A β 1-42 can seed the aggregation of A β 1-40.

The APP gene was sequenced and found to be encoded on chromosome 21. Expression of the APP gene generates several A β-containing isoforms of 695, 751 and 770 amino acids, with the latter two APPs containing a domain that shares structural and functional homologies with Kunitz serine protease inhibitors (Kang et al., 1987; Kitaguchi et al., 1988; Ponte et al., 1988; Tanzi et al., 1988; Konig et al., 1992). The functions of APP in the nervous system remain to be defined, although there is increasing evidence that APP has a role in mediating adhesion and growth of neurons (Schubert et al., 1989; Saitoh et al., 1994; Roch, 1995) and possibly in a G protein-linked signal transduction pathway (Nishimoto et al., 1993). In cultured cells, APPs mature through the constitutive secretory pathway (Weidemann et al., 1989; Haass et al., 1992; Sisodia 1992) and some cell-surface-bound APPs are cleaved within the A β domain by an enzyme, designated α-secretase, (Esch et al., 1990), an event that precludes A β amyloidogenesis. Several studies have delineated two additional pathways of APP processing that are both amyloidogenic: first an endosomal/lysosomal pathway generates a complex set of APP-related membrane-bound fragments, some of which contain the entire A β sequence (Haass et al., 1992; Golde et al., 1992); and second, by mechanisms that are not fully understood, A β 1-40 is secreted into the conditioned medium and is present in cerebrospinal fluid in vivo (Haass et al., 1992; Seubert et al., 1992; Shoji et al., 1992; Buscigho et al., 1993). Lysosomal degradation is no longer thought to contribute significantly to the production of A β (Sisodia and Price 1995). The proteolytic enzymes responsible for the cleavages at the NH2 and COOH termini of A β are termed β (BACE) and γ. secretase, respectively. Until recently, it was generally believed that A β is generated by aberrant metabolism of the precursor. The presence, however, of A β in conditioned medium of a wide variety of cells in culture and in human cerebrospinal fluid suggest that A β is produced as a normal function of cells.

The main focus of researchers and the principal aim of those associated with drug development for AD is to reduce the amount of A β deposits in the central nervous system (CNS). These activities fall into several general areas: factors affecting the production of A β, the clearance of A β, and preventing the formation of insoluble A β fibrils. Another therapeutic goal is to reduce inflammatory responses evoked by AP neurotoxicity. Several groups have demonstrated the ability of the Alzheimer's disease toxin, A β 1-42, to induce antibody titers in either wild-type, APP, or APP/PS1 transgenic mice (Schenk et al. 1999, Janus et al. 2000, Morgan et al. 2000). Sufficient immunization with peptide also leads to reduction in amyloid burden and improved cognition in transgenic mice. Apparently, more than one mechanism contributes to antibody efficacy, including sequestering of A β peptides in the periphery and induction of Fc-γ receptor mediated phagocytosis by microglia in the brain. Frangionne et al., (PCT/US01/16322) demonstrated that a shortened version of the A β 1-42 toxin can also to induce antibodies and reduce amyloid burden in a transgenic model of AD. This peptide includes the first 30 amino acids of A β 1-42 plus a N-terminal tail of six lysine residues; it has the added advantage of not being fibrillogenic or cytotoxic in vitro. Additional modifications to the 1-30 amino acid peptide have been proposed, including substitutions at amino acids 17-21 and N- or C-terminal additions, that will confer both reduced fibrillogenicity/toxicity and improved immunogenicity in the vaccinated host.

The immune response to viral infections of the CNS is probably initiated in peripheral lymphoid tissue followed by entry of activated T cells into the cerebrospinal fluid, meninges, and brain parenchyma (Griffin, et al. 1992). Full development of the inflammatory response requires virus-specific T cells, while additional participating cells include NK cells, monocytes and B cells. Likewise, in Rasmussen's encephalitis, it was recently shown that a cytotoxic T-cell mechanism contributes to loss of neurons in human brain disease (Bien, et al. 2002). Immunohistochemical evaluation of specimens from these patients revealed lymphocytic infiltrates that consisted mainly of CD3(+)CD8(+) T cells, some of which lay in direct apposition to MHC class I(+) neurons. Likewise, in diseases of putative autoimmune background, such as ADLE or MS, the patterns of brain inflammation are characterized by T-cell inflammation with macrophage and microglia activation, the majority of infiltrating T cells in the lesions being CD8+and class I restricted (Gay et al. 1997).

There is a need for a method to screen sequences of amyloid beta peptides or homologues thereof for identifying T-cell epitopes, to the amyloid beta peptides which lack T-cell epitopes and to a vaccine comprising amyloid beta or a homologue thereof by selecting peptide which lacks T-cell epitopes or in which at least one amino acid was deleted or changed. Further, there is also a need for predicting the reaction of an individual to a vaccine which comprises amyloid beta peptide or homologue thereof for immunization against Alzheimer's Disease or other diseases of amyloid beta accumulation.

### BRIEF DESCRIPTION OF THE DRAWINGS

FIG. 1: FIG. 1: Binding of radiolabelled peptide to 1 nM rHLA A0201 in absence or presence of 1 uM Abeta 1-42 or homologue-derived peptides (numbered 1-10; see Table 4). Binding is shown relative to measured binding without competition (maximal binding). The control peptide (ctrl): FLPSDYFPSV (SEQ ID NO. 1).

FIG. 2: Binding of radiolabelled peptide to 1 nM rHLA A0201 in increasing doses of Abeta 1-42 or homologue-derived peptide epitopes (numbered 1-10; see Table 4). Binding is shown relative to measured binding without competition (maximal binding). The control peptide (ctrl): FLPSDYFPSV (SEQ ID NO. 1)

FIG. 3: Binding of radiolabelled peptide to 1 nM rHLA A0201 in increasing doses of Abeta 1-42 or homologue-derived peptide epitopes (numbered 1-10; see Table 4). Binding is shown relative to measured binding without competition (maximal binding). The control peptide (ctrl): FLPSDYFPSV (SEQ ID NO. 1). IC50 values and Hill coefficients were calculated from binding data fitted to inhibition curves using GraphPad Prism 3.0.

### SUMMARY OF THE INVENTION

In one embodiment of the invention, there is provided an isolated amyloid beta peptide or homologue thereof, which lacks or has reduced ability to induce harmful T-cell response, and the vaccine comprising the same for the prevention or treatment of Alzheimer's Disease.

In another embodiment, the invention provides a vaccine comprising an amyloid beta peptide or homologue thereof and a carrier or a diluent, wherein the amyloid beta peptide or homologue thereof lacks or has reduced ability to induce an undesirable T-cell response.

In one embodiment, the invention provides a method of determining T-cell epitopes within amyloid beta peptide or homologue thereof comprising the steps of: a. determining the binding value of each amino acid of a subsequence of amyloid beta peptide or homologue thereof upon binding to a HLA class 1 and/or class II molecule of interest; b. determining the resulting score of amino acids of the subsequence based on the binding value of amino acids obtained in step a; and c. comparing the resulting score to a preselected value, to predict the presence of T-cell epitopes within amyloid beta peptide or homologue thereof.

In another embodiment, the method relates to an isolated amyloid beta peptide or homologue thereof, wherein the peptide or homologue are selected according to the method comprising the steps of; a. determining the binding value of each amino acid of a subsequence of amyloid beta peptide or homologue thereof upon binding to a HLA class 1 and/or class II molecule of interest; b. determining the resulting score of amino acids of the subsequence based on each of the binding value of each amino acid obtained in step a; and c. comparing the resulting score to a preselected value, wherein a subsequence with a resulting score, which is less than the preselected value is then selected to be contained within the isolated amyloid beta peptide or homologue thereof.

In another embodiment, the invention provides a vaccine comprising an amyloid beta peptide or homologue thereof, wherein the peptide or homologue thereof are selected according to the method comprising the steps of: a. determining the binding value of each amino acid of a subsequence of amyloid beta peptide or homologue thereof upon binding to a HLA class 1 and/or class II molecule of interest; b. determining the resulting score of all amino acid of the subsequence based on the binding value of each amino acid obtained in step a; and c. comparing the resulting score to a preselected value, wherein a subsequence with a resulting score, which is less than the preselected value is then selected as contained in the isolated amyloid beta peptide or homologue thereof of the vaccine.

In another embodiment, the invention provides a method of predicting the reaction of an individual to a vaccine, which comprises amyloid beta peptide or homologue thereof, comprising the following steps: a. obtaining a sample from a subject; b. determining the HLA haplotype of the subject; c. determining the binding value of each amino acid of a subsequence of amyloid beta peptide or homologue thereof to HLA haplotype of the individual; d. determining the resulting score of all amino acid of the subsequence based on the binding value of each amino acid obtained in step c; and; e. comparing the resulting score to a preselected value, wherein if the resulting score is higher than the preselected score, the individual has the potential to develop T-cell responses, and if the resulting score is lower than the preselected score the individual does not have the potential to develop T cell responses.

In another embodiment, the invention provides a method of matching a vaccine comprising a beta amyloid or homologue peptide thereof to an individual, for immunization of an individual, based on the HLA haplotype of the individual comprising: a. obtaining a sample from a subject; determining the HLA haplotype of the subject; c. determining the binding value of each amino acid of a subsequence of amyloid beta peptide or homologue thereof to HLA haplotype of the individual; d. determining the resulting score of all amino acids of the subsequence based on the binding value of each amino acid obtained in step c; and e. comparing the resulting score-to a preselected value, wherein if the resulting score is lower than the preselected score, the amyloid beta peptide or homologue thereof is suitable for preparing a vaccine comprising beta amyloid peptide or homologue thereof for immunization of an individual.

In another embodiment, the invention provides a kit for matching a vaccine comprising amyloid beta peptide or homologue thereof to an individual based on the HLA haplotype of the individual comprising: a) a means for obtaining a blood sample from the individual; b) a means for determining the HLA haplotype of the individual; and c) a means for determination of the binding of subsequence of amyloid beta or homologue to HLA haplotype of the individual.

In another embodiment, the invention provides a vaccine comprising an amyloid beta peptide or homologue thereof, wherein the amyloid beta peptide or homologue thereof lacks the ability to induce a T-cell response.

In another embodiment, the invention provides an amyloid beta peptide or homologue thereof, wherein the amyloid beta peptide or homologue thereof, lacks the ability to induce a T-cell response.

In another embodiment, the amyloid beta peptide or homologue thereof, which is selected by its lack of its ability to induce a T-cell response and the vaccine comprising the same, are used for the prevention of amyloid beta plaque formation.

Description of the Detailed Embodiments

Vaccination with Aβ and Aβ homologs i.e. from the same species (with more than 70% homology to the amyloid beta peptide) has been proven efficacious in transgenic models of Alzheimer's disease. However, in light of the recent reports of cerebral inflammation as a detrimental side effect of an A β vaccine trial, additional safety issues must be considered and appropriate modifications incorporated into the vaccine antigen. The homologs proposed by Sigurdsson et al. (WO0190182 and WO 03/045128 A2) include truncations of the wild-type peptide at residue 30, C- and N-terminal additions, and internal modifications at residues 17-21. These homologs are less likely to form P-sheets and toxic fibrils, while still able to induce an antibody response to the wild-type toxic A β peptide.

The present invention describes the selection of an amyloid beta peptide or homolog thereof and a vaccine comprising the same which comply with at least one of the following criteria: 1) the antigen will be less likely to cause an autoimmune response in patients; 2) the antigen will retain its ability to mount a productive immune response in the host; 3) the antigen will have a reduced ability to form toxic fibrils. The present invention also describes additional point modifications to the selected peptides to even further reduce their toxicity in terms of T-cell autoimmune response, while retaining their ability to induce a productive antibody response in the patient. In one embodiment of the invention, there is provided an isolated amyloid beta peptide or homologue thereof, which lack or has reduced ability to induce harmful T-cell response and the vaccine comprising the same, are used for the prevention or treatment of Alzheimer's Disease.

In another embodiment, the invention provides a vaccine comprising an amyloid beta peptide or homologue thereof and a carrier or a diluent, whereby the amyloid beta peptide or homologue thereof lack or have reduced ability to induce an undesirable T-cell response.

The terms “amyloid beta”, or “Aβ”, or “amyloid β”, or “beta amyloid” are all referred to interchangeably hereinabove to any of the amyloid β species. Such proteins are typically of about 4 kDa, but can be less or more. Several different amino-termini and heterogeneous carboxyl-termini sequences have been observed based on characterization of the peptide amyloid β from Alzheimer's disease tissue and from cultured cells (Glenner and Wong (1984; Joachim et al. (1988); Prelli et al. (1988); Mori et al. (1992); Seubert et al. (1992); Naslund et al. (1994); Roher et al. (1993); Busciglio et al. (1993); Haass et al. (1992)). Specifically, with regard to the carboxyl-termini, the amyloid β peptide has been shown to end at position 39, 40, 41, 42, 43, or 44 where position 1 is the aspartate of the amyloid β sequence as defined by Glenner et al. 1984.

While recognizing the dominant role of full-length Aβ peptides, the present invention is not limited solely to these forms. Thus, notwithstanding the importance of full-length Aβ peptides as major therapeutic targets, the invention also envisages using subsequences of amyloid beta i.e amyloid β fragment or truncated amyloid beta or heterogeneous amyloid β as immunogens. The term “immunogen” refers hereinafter to a substance capable of inducing an immune response (as well as reacting with the products of an immune response).

The terms “amyloid β fragment” or “heterogeneous amyloid β” or “truncated amyloid β” interchangeably refer to fragments derived from the full length beta amyloid peptide defined above. Biochemical studies have demonstrated that in addition to an L-aspartate at positions 1, Aβ peptides can begin with a racemized or isomerized aspartate. Prominent N-terminus truncated Aβ isoforms begin with a cyclized glutamate (pyroglutamate) residue at position 3, pyroglutamate at position 11, and leucine at position 17 (Geddes et al 1999). Support for the fact that these isoforms contribute to the pathogenesis of Alzheimer's Disease is also based on studies which demonstrate 1) N-terminus truncated forms aggregate more readily and are more toxic in vitro than Aβ1-40 or Aβ1-42 (Pike et al. 1995) and 2) N-terminus truncated forms are among the earliest isoforms detected in plaques and may form a nidus for plaque formation (Tekirian, 2001). Aβ17-42 (the p3 peptide) for example, is prevalent in AD brains but absent or sparse in aged, non-AD brains (Higgins et al. 1996). Studies of AD amyloid with high-resolution reverse-phase liquid chromatography and mass spectrometry confirm that additional N-terminus truncated forms are invariably present, including Aβn-42 (n=1-11) and Aβ3-40 (Lamer 1999). Studies of Aβ secreted into media of various cultured cells and cell lines transfected with differing APP constructs have identified Aβ species beginning at positions 2, 3, 4, 5, 6, 9, 11, 16, 17, 18, 19, 20, 24 (Busciglio et al 1993, Haas et al 1992, Haas et al 1994). The “nonamyloidogenic” p3 fragment (amyloid beta 17-42) is a major constituent of Down's syndrome cerebellar preamyloid (Lalowski M et al. 1996). A vaccination which includes major forms, or limiting its neurotoxicity, can therefore be expected to slow progression of Down syndrome-associated Alzheimer's Disease and delay onset in susceptible individuals.

In another embodiment, the invention provides a composition comprising the amyloid beta peptide or homolog thereof which lack or have reduced ability to induce T-cell response and an acceptable pharmaceutical carrier.

In anther embodiment, the invention provides a vaccine comprising the amyloid beta peptide or homolog thereof and a diluent or a carrier, whereby the peptide or homolog thereof lack or have reduced ability to induce T-cell response.

In one embodiment, the invention provides a method of determining T-cell epitopes within amyloid beta peptide or homologue thereof comprising the steps of: determining the binding value of each amino acid of a subsequence of amyloid beta peptide or homologue thereof upon binding to a HLA class I and/or class II molecule of interest; the binding value of the amino acid can be represented according to one embodiment of the invention, as the contribution of this amino acid to the half life time for disassociation of the subsequence to the HLA class I and/or class II molecule. It should be noted that the binding value of a specific amino acid may be varied according to its position in the sequence and according to the ‘neighboring’ amino acids; determining the resulting score of all amino acid of the subsequence based on the binding value of each amino acid obtained in the previous step; and comparing said resulting score to a preselected value, to predict presence of T-cell epitopes within amyloid beta peptide or homologue thereof. The term “T-cell” refers hereinafter to a type of lymphocyte. T cells have T-cell receptors and, sometimes, co-stimulatory molecules on their cell surfaces. The T cell helps to orchestrate the immune system and can induce other cells to make cytokines and chemokines. The term “T-cell epitope” refers hereinafter to a single antigenic determinant. Functionally it is the portion of an antigen which combines with the antibody or T-cell receptor. By the term “antigen” or “antigenic determinant” is something recognized by the immune system (usually foreign proteins).

The term “lack” refers herein to either does not have the ability or to reduced ability i.e where the response is not leading to cell death or damage, according to known methods of the art. As is known to those skilled in the art, one way to identify the regions which can bind to MHC and -evoke a T cell response is to scan the whole antigen sequence by synthesizing overlapping peptide fragments and assaying for immune reactions.

MHC binding peptide prediction methods can be divided into three main groups a) Motif based methods, b) Statistical/Mathematical expression based methods and, c) Structure based methods. Binding motifs describe general position based patterns of recurrent amino acids favorable for HLA-peptide binding. Prediction methods based on binding motifs are mostly all or none algorithms with high false rates. Statistical/Mathematical expression based methods include Quantitative matrix and Neural network based methods. Quantitative matrices provide a linear model with easy to implement capabilities.

Their predictive accuracies are considerable. On the other hand, neural networks are more complex, nonlinear and self learning systems. Their predictive accuracies are higher but they require large amount of data for learning which makes Quantitative matrix based methods suitable for MHC binding peptide predictions. Structure based methods are logically very sound but computationally complex. These methods calculate binding energy of peptide-MHC complex and the energetically favorable peptides are predicted as binders. These methods are in stages of development. All the above mentioned approaches cannot effectively deal with MHC Polymorphism i.e. for each allele a separate matrix has to be generated or a separate set of rules have to be applied. Recently, Stumiolo et al., 1999 provided an answer by using virtual matrix which holds promise for delivering better MHC binding peptide prediction method. Publicly accessible algorithms from the BioInformatics & Molecular Analysis Section (BIMAS) of the National Institutes of Health rank potential peptides based on predicted half-time of dissociation to HLA class I molecules. They are based on coefficient tables deduced from the published literature by Dr. Kenneth Parker (Parker 1994), Applied Biosystems (see website http://bimas.dcrt.nih.gov/molbio/hla_bind/). Additional programs and databases that could be used for prediction of epitopes for both class I and/or class II molecules are found, for example, at the SYFPEITHI website (http://syfpeithi.bmi-heidelberg.com/scripts/MHCServer.dll/home.htm) and the HIV Molecular Immunolgoy Database website (http://hiv.basic.nwu.edu/HLA/MotifScanner.cfm) and the Molecular Immunology Foundation Tools for Science website—RANKPEP (http://mif.dfci.harvard.edu/Tools/). The step of determining the resulting score of all amino acid of the subsequence based on each of the binding value of each amino acids obtained in step a is conducted by addition of each of the amino acid values and by simply adding the values or multiplication. In another embodiment, the determining step so as to obtain a resulting score can be performed by using a complex mathematical function. The resulting score is compared to preselected value or preselected score, to predict presence of undesirable T-cell epitopes within amyloid beta peptide or homologue thereof.

The term “preselected score” refers hereinafter to a value, which represents a threshold value. Any value which is lower than that value represents subsequences with low probability of inducing T-cell responses. Any number which is higher than this value predicts the presence of a T-cell epitope which may induce T-cell responses (for example without being limited Example 6, Table 7 SEQ ID No. 133 and 134 have scores higher than the threshold of 49.00).

In another embodiment, the invention provides a vaccine comprising an amyloid beta peptide or homologue thereof, wherein the peptide is selected according to the method comprising the steps of: a. determining the binding value of each amino acid of a subsequence of amyloid beta peptide or homologue thereof for binding to a HLA class I and/or class II molecule of interest; the subsequence includes, without being limited, 8-12 amino acids for class I, and usually, but not limited to 15 amino acids for class II; b. determining the resulting score of all amino acids of the subsequence based on the binding value of each amino acid obtained in step a; and c. comparing said resulting score to a preselected value, wherein subsequence with a resulting score which is less than said preselected value is then selected as contained in the isolated amyloid beta peptide or homologue thereof of the vaccine.

In one embodiment, the invention provides a method to identify an isolated amyloid beta peptide or homologue thereof for use as immunogens. The invention enables selection of amyloid beta peptide or homologue thereof, which will contain an amount of T-cells epitopes which will not induce undesirable T-cell responses. In another embodiment, the invention describes Aβ-derived peptides for human vaccination which have been modified by certain amino acid substitutions and/or additions in order to remove or reduce undesirable T-cell epitopes. These epitopes are defined by their ability to bind HLA molecules according to previously published methods. These epitopes are further defined by their ability to elicit T cell responses such as T cell proliferation or cytotoxicity in human lymphocytes in vitro. In another embodiment, the peptides contain modifications that reduce their fibrillogenicity and toxicity in vitro and also remove potentially undesirable T-cell epitopes.

In another embodiment, the invention provides peptides that are selected according to the above described method of selecting a peptide. The peptides selected according to the above described methods are further assessed in vitro or in vivo in laboratory animals for lack of undesirable T-cell response. The tests conducted of which some are provided in details in the Examples section are well known in the art and are used to identify the peptides that do not cause proliferation of T-cells. In another embodiment, the peptide is assessed for lack of ability to induce cytotoxicity. i.e. to induce cell killing by the T-cells. In another embodiment, the selected peptides are assessed for their lack of ability to secrete cytokines. The term “lack” is refers herein to either lack or to reduced ability i.e where the response is not leading to cell death or damage, according to known methods of the art.

In another embodiment, the peptides are assessed for fibrillogenicity and for lack of ability to form a beta sheet structure, which can lead to aggregation of amyloid beta and to formation of amyloid plaques (see in the Examples section.). In another embodiment, the peptide is further assessed for lack of toxicity. For example, it does not cause increase in the amount of free radicals or interact with certain cell-surface receptors involved toxic pathways (see in the Examples section). In another embodiment, the peptide is further assessed for lack of cytotoxicity, i.e. it does not cause cell death (see in the Examples section).

In another embodiment, the peptide is examined for its ability to induce antibody response, for example, by repeated administration of amyloid beta peptides or homologue thereof into wild-type or APP transgenic mice, or into guinea pigs (which have the same amino acid sequence for Aβ as do humans) and determination of antibody titers against the endogenous Aβ toxin, using for example standard ELISA testing.

In another embodiment, the selected peptide is further assessed for its ability to bind to MHC class II molecule of interest so as to predict the ability of the selected peptide to activate T-helper cells. The method is similar to the method described above for the HLA class I cells. In particular, if the peptide or homologue is combined or delivered with another molecule that can provide T-cell help to the host, it may be advantageous to remove endogenous T-helper epitopes from the peptide or homologue of AP.

In another embodiment, the invention provides a vaccine comprising an amyloid beta peptide or homologue thereof, whereby the amyloid beta peptide or homologue thereof lacks the ability to induce an undesirable T-cell response. According to this embodiment, the peptides are selected by biological methods, in vitro methods as well as in vivo methods, as described before for the peptides selected according to the computerized methods.

Although the MHC molecule expression frequency distribution can vary across different ethnic groups, it may be theoretically possible to remove detrimental T-cell epitopes for greater than 90% of a given population by identifying epitopes associated with the six most prevalent class I MHC molecules in the population. MHC or HLA can be used hereinafter interchangeably—The major histocompatibility complex of humans (denoted HLA-human leukocyte antigen) is a cluster of genes occupying a region located on the sixth chromosome. MHC-I Major Histocompatibility Complex Class I comprise HLA-A,B,C tissue type. MHC-II Major Histocompatibility Complex Class II, HLA-DR, -DQ, and -DP proteins contain two polymorphic chains, designated alpha and beta. These D-region proteins are encoded by loci designated DRA, DRB1, DRB3, DRB4, DQA1, DQB1, DPA1, and DPB1.

However, it may be important to screen individuals before treatment to determine the safety of the vaccine antigen as it relates to their particular genotype. In one embodiment, this invention describes a method for screening individuals for their HLA haplotype in order to assess their suitability for vaccine treatment.

As used herein, “haplotype” is a region of genomic DNA on a chromosome which is bounded by recombination sites such that genetic loci within a haplotypic region are usually inherited as a unit. However, occasionally, genetic rearrangements may occur within a haplotype. Thus, the term haplotype is an operational term that refers to the occurrence on a chromosome of linked loci.

Screening can be done using standard techniques of the art, or those that are developed subsequently. For example, in addition to the traditional, serological methods of typifying HLA, a series of DNA analysis methods have been described. Based on the polymerase chain reaction, a certain allele can be typified by amplification with sequence-specific primers (SSP-PCR), by hybridization with sequence-specific oligonucleotides (SSOP-PCR) or by the use of restriction length polymorphism. The disadvantages of serological typification are that living cells are needed for the test, and that there is a possibility of false interpretation caused by cross-reactivity between the alloantisera and monoclonal antibodies. On the other hand, typification by polymerase chain reaction has proved to be fundamentally more exact and reliable. The individual samples are also easier to store and transport, and can be tested repeatedly.

One such method involves the use of DNA restriction fragment length polymorphism (RFLP) as a basis for HLA typing. See Erlich U.S. Pat. No. 4,582,788, issued Apr. 15, 1986. Polymorphism detected by this method is located in both coding and noncoding sequences of the genome. Therefore, RFLP often does not directly measure functional polymorphism, but relies upon linkage disequilibrium between polymorphism in non-coding regions and the coding region. RFLP analysis has been used for typing an HLA-deficient severe combined immunodeficiency (SCID) patient, but its utility as a routine method is limited by laborious procedures, inadequate resolution of alleles, and difficulty in interpreting data for certain combinations of alleles. Some RFLP and similar typing methods utilize labelled obligonucleotides to identify specific HLA and DNA sequences. In particular, the use of oligonucleotide probes have been found advantageous in HLA-DR typing in identifying variant genes encoding products which are not detectable serologically. See Angelini et al., above, Scharf et al., Science, Vol. 233, No. 4768, pp. 1076-1078, Cox et al., Am. J. Hum. Gen., 43:954-963, 1988, Tiercy et al., Proc. Natl. Acad. Sci. USA, Vol. 85, pp. 198-202, 1988, and Tiercy et al., Hum. Immunol. 24, pp. 1-14 (1989). Sequence-specific oligonucleotide probe hybridization (SSOPH) can discriminate single base pair mismatches, which is equivalent to detecting a single amino acid polymorphism in HLA proteins.

The polymerase chain reaction ICR) process, as described in Mullis U.S. Pat. No. 4,683,202, issued Jul. 28, 1987, allows the amplification of genomic DNA and has given rise to more convenient BLA typing procedures. HLA-DQ alpha and HLA-DP alpha and beta genes have been amplified, and then sequenced or hybridized with oligonucleotide probes. See Saild et al., Nature, Vol. 324, pp. 163-166, 1986, Bugawan et al., J. Immunol., Vol. 141, No. 12, pp. 4024-4030, 1988, and Gyllensten et al., Proc. Natl. Acad. Sci. USA, Vol. 85, pp. 7652-7656, 1988.

Once a subject haplotype is known, a vaccine treatment can be initiated accordingly. The invention provides a method of matching a vaccine comprising a beta amyloid or homologue peptide thereof to an individual, for immunization of an individual based on the HLA haplotype of the individual. A method of matching a vaccine comprising a beta amyloid or homologue peptide thereof to an individual, for immunization of an individual wherein the based on the HLA haplotype of the individual comprising: a. obtaining a sample from a subject; determining the HLA haplotype of said subject; c. determining the binding value of each amino acid of a subsequence of amyloid beta peptide or homologue thereof to HLA molecules of said individual; d. determining the resulting score of all amino acids of the subsequence based on each of the binding value of each amino acids obtained in step a; and comparing said resulting score to a preselected value, wherein if said resulting score is lower than said preselected score, the beta amyloid or homologue thereof is selected for preparing a vaccine comprising beta amyloid peptide or homologous thereof for immunization of an individual based on the haplotype of the individual and if said resulting score is higher than said preselected score, the beta amyloid or homologue thereof is not selected for immunization of the individual based on the haplotype of the individual.

Certain peptides will have the similar antibody-stimulating potential, but include different modifications to remove T-cell epitopes that may be harmful to the particular individual. An individual may be deemed a candidate for vaccine therapy based on the results of this screening procedure. A certain individual may be denied such treatment because of the likely event of a T-cell mediated autoimmune response. This screening procedure will enhance the safety of any vaccine program for Alzheimer's disease.

Mendelian genetics states that the frequency of alleles at one locus do not influence the frequency of alleles at another locus. However in HLA genetics this is not true. There are a number of examples from within the HLA system of alleles at different loci occurring together at very much higher frequencies than would be expected from their respective gene frequencies. This is termed linkage disequilibrium.

Because of linkage disequilibrium, a certain combination of HLA Class I antigen, HLA Class II antigen and Class HI products will be inherited together more frequently than would normally be expected. It is possible that these “sets” of alleles may be advantageous in some immunological sense, so that they have a positive selective advantage. Linkage disequilibrium may also be important for understanding an individual's response to a certain antigen and a screening procedure may also allow for identification of combinations of HLA alleles that have a preferred or reduced ability to respond to an Abeta vaccine antigen.

In another embodiment, this screening method can be applied to vaccine therapies for other diseases where the antigen administered is a self-antigen. In most cases, the self-antigen is designed to elicit an antibody response, but a cytotoxic, or a helper T-cell response would be undesirable. A treatment regimen could be initiated or not depending on the results of the screening program.

Peptides of other self-antigens designed for use in a vaccine therapy can be modified accordingly in order to remove undesireable prominent T-cell epitopes. Patients will receive vaccine treatment by matching the modified peptide to their personal haplotype. In all cases, the modifications will reduce potency or remove T-cell epitopes but not destroy the important antibody-inducing antigenic epitopes of the peptide. In preferred instances, the modifications will also reduce or eliminate additional detrimental motifs of the self antigen. An unlimiting example is the modification of Abeta to reduce it fibrillogenicity and toxicity and to remove harmful T-cell epitopes, while retaining its ability to induce an antibody response in vivo.

The same strategy can be applied to other vaccine self-antigens that demonstrate β-sheet structure and protein aggregation. Examples of disease-forming proteins that may be used for vaccine purposes include: prion protein, amylin, α-synuclein, and polyglutamine repeats. In a U.S. provisional application, Sigurdsson et al. disclosed vaccination of individuals with diseases-specific peptide homologs, which have been modified to demonstrated reduced fibrillogenicity and toxicity in vitro. In order to ensure the safety of these vaccines, modifications will be made that not only reduce their aggregation status, but also remove detrimental T-cell epitopes which could result in an autoimmune reaction in the patient. Likewise, the use of a screening method to determine the suitability of an individual for a certain vaccine antigen is disclosed.

In another embodiment, the invention provides a kit for matching a vaccine comprising amyloid beta peptide or homologous thereof to an individual based on the HLA haplotype of the individual comprising of: a) a means for obtaining a sample from the individual; The sample can be a body fluid such as blood or CSF or can be a tissue such as without being limited skin or nose epithelium. b) a means for determining the HLA haplotype of the individual; these may be one or more of the reagents used in the above described methods for determination of the haplotype of the individual. For example, without limitation in one embodiment, the kit comprises at least one genetic locus-specific primer pair in a suitable container. The primers of each pair can be in separate containers, particularly when one primer is used in each set of primer pairs. However, each pair is preferably provided at a concentration which facilitates use of the primers at the concentrations required for all amplifications in which it will be used. The kit may further contain means for determination of the binding of subsequence of amyloid beta or homologue to HLA haplotype of the individual. These can be either a table, which gives value to what will be the binding value of a specific amyloid beta peptide or homologue or it could be a programmed calculator, where a person skilled in the art can enter the specific amyloid beta sequence of interest or homologue thereof. The kit can serve either for matching a specific amyloid beta sequence to a vaccine for a specific individual, or can be used for predicting the reaction of the individual to a specific amyloid beta peptide.

In another embodiment the invention provides a method for the treatment or prevention of Alzheimer's Disease, wherein the method comprising the step of administering amyloid beta fragment or homolog thereof, which lacks the ability to induce undesirable T-cell response.

In another embodiment the invention provides a method for the treatment or prevention of Alzheimer's Disease, wherein the method comprising the step of administering a vaccine comprising amyloid beta fragment or homolog thereof, which lacks the ability to induce undesirable T-cell response.

In another embodiment the invention provides a method for preventing amyloid plaque formation, wherein the method comprising the step of administering amyloid beta fragment or homolog thereof, which lacks the ability to induce undesirable T-cell response.

In another embodiment the invention provides a method for preventing amyloid plaque formation, wherein the method comprising the step of administering a vaccine comprising amyloid beta fragment or homolog thereof, which lacks the ability to induce undesirable T-cell response.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

Epitope Identification:

To identify T-cell epitopes, one can scan the sequences of peptides to find regions containing the known epitope-binding motif for class I or class II HLA alleles. Motifs are then synthesized as peptides of 8-11 (class I) or around 15 (class H) amino acids and tested for immunogenicity, using a variety of techniques as detailed below, in human peripheral blood lymphocytes.

### Example 1

The sequence of Ab1-43 and the sequence of 1-30VF/EE i.e the 1-30 amyloid beta peptide, wherein the VF was replaced by EE, were entered into the HLA Peptide Binding Prediction program at BIMAS using the subsequence length of 9 amino acids. The results were analyzed for all possible HLA Class I options (32 alleles) listed on the program home page. The results can be classified into three categories: a) epitopes which do not exist in the 1-30VF/EE peptide because they require residues between amino acids 31-43. When analyzing the top 10-ranked epitopes, 40-80% of the epitopes were eliminated in all of the 32 HLA alleles. Thus, a significant proportion of detrimental T-cell epitopes do not exist in the shortened homolog; b) epitopes that have a reduced score or are eliminated due to the internal modifications of EE at positions 18 and 19; c) epitopes that have an increased score or are added as a result of the internal modifications of EE at positions 18 and 19. Tables 1 and 2 are exemplary of this type of analysis, performed on the most prevalent HLA molecule found in the Caucasian population. Comparison of these two tables shows that seven epitopes which are present in the Abeta 1-43 sequence (at start positions 33, 34, 31, 35, 28, 32, and 24) do not appear in the analysis of the 1-30VF/EE peptide. Of those seven sequences, at least three have a score high enough to be assumed significant. The epitope starting at position 16 has a score of 453.27 in the Abeta 1-43 peptide, which is decreased almost 4-fold to 119.938 in the 1-30VF/EE peptide, due only to the change of residues VF to EE. Likewise, the epitope starting at position 10 has a score of 6.221 in the Abeta 1-43 peptide. This score is reduced to 0.001 in the 1-30VF/EE peptide and can be considered negligible in terms of its contribution to a possible T-cell response. No epitopes were improved or added in the 1-30VF/EE peptide. In summary, the 1-30VF/EE antigen contains both fewer and lower-scored A_{—}0201 epitopes than the Abeta 1-43 antigen. This suggests a greatly reduced probability of mounting a harmful T-cell response to the 1-30VF/EE antigen in patients with this haplotype.

TABLE 1
Analysis of peptide predictions based on
binding of subsequences from Abeta 1-43
(DAEFRHDSGYEVHHQKLVFF
AEDVGSNKGAIIGLMVGGVVIAT
SEQ ID No. 2)_0201 molecule.

| | | | Score (Estimate of Half Time of |
|---|---|---|---|
| | Start | Subsequence Residue | Disassociation of a Molecule |
| Rank | Position | Listing | Containing This Subsequence) |
| 1 | 16 | KLVFFAEDV | 453.270 |
| | | SEQ ID NO. 3 | |
| 2 | 33 | GLMVGGVVI | 15.827 |
| | | SEQ ID NO. 4 | |
| 3 | 10 | YEVHHQKLV | 6.221 |
| | | SEQ ID NO. 5 | |
| 4 | 34 | LMVGGVVIA | 5.752 |
| | | SEQ ID NO. 6 | |
| 5 | 31 | IIGLMVGGV | 4.861 |
| | | SEQ ID NO. 7 | |
| 6 | 35 | MVGGVVIAT | 2.550 |
| | | SEQ ID NO. 8 | |
| 7 | 28 | KGAIIGLMV | 1.589 |
| | | SEQ ID NO. 9 | |
| 8 | 4 | FRHDSGYEV | 0.182 |
| | | SEQ ID NO. 10 | |
| 9 | 32 | IGLMVGGVV | 0.152 |
| | | SEQ ID NO. 11 | |
| 10 | 24 | VGSNKGAII | 0.047 |
| | | SEQ ID NO. 12 | |

TABLE 2
Analysis of peptide predictions based on binding
of subsequences from Abeta 1-30VF/EE
(DAEFRHDSGYEVHHQKLE
EFAEDVGSNKGA-SEQ ID No. 13, 0201 molecule).

| | | | Score (Estimate of Half Time of |
|---|---|---|---|
| | Start | Subsequence Residue | Disassociation of a Molecule |
| Rank | Position | Listing | Containing This Subsequence) |
| 1 | 16 | KLEEFAEDV | 118.938 |
| | | SEQ ID NO. 14 | |
| 2 | 4 | FRHDSGYEV | 0.182 |
| | | SEQ ID NO. 15 | |
| 3 | 2 | AEFRHDSGY | 0.005 |
| | | SEQ ID NO. 16 | |
| 4 | 10 | YEVHHQKLE | 0.001 |
| | | SEQ ID NO. 17 | |
| 5 | 8 | SGYEVHHQK | 0.001 |
| | | SEQ ID NO. 18 | |
| 6 | 15 | QKLEEFAED | 0.001 |
| | | SEQ ID NO. 19 | |
| 7 | 21 | AEDVGSNKG | 0.001 |
| | | SEQ ID NO. 20 | |
| 8 | 22 | EDVGSNKGA | 0.001 |
| | | SEQ ID NO. 21 | |
| 9 | 18 | EEFAEDVGS | 0.000 |
| | | SEQ ID NO. 22 | |
| 10 | 13 | HHQKLEEFA | 0.000 |
| | | SEQ ID NO. 23 | |

### Example 2

Similar analysis can be performed on additional Abeta homologs, with alternative substitutions that also are predicted to decrease fibrillogenicity and toxicity. Table 3 shows the top five ranked peptides for several of these modified peptides (b-f) and compares them to the top five ranked peptides for Abeta 1-30 (a). Changes such as LV/EE, LV/DD, and LV/KK render the two major epitopes of Abeta 1-30, starting at positions 16 and 10, irrelevant (Table 3b-d). The score for these epitopes has dropped to below 0.6 in the three modified peptides. The LVF/EEE and LVF/EDD peptides both lose the epitope starting at position 10, but will likely retain significant binding of the position 16 epitope to HLA A_{—}0201 molecules (Table 3e-f).

TABLE 3
Analysis of peptide predictions based on
binding of subsequences from Abeta 1-30 homologs
to the HLA A_0201 molecule.

| | | | |
|---|---|---|---|
| | | | Score (Estimate of Half Time of |
| | Start | Subsequence Residue | Disassociation of a Molecule |
| Rank | Position | Listing | Containing This Subsequence) |

a) Sequence
DAEFRHDSGYEVHHQKLVFFAEDVGSNKGA,
SEQ ID NO. 24 (no modifications)

| | | | |
|---|---|---|---|
| 1 | 16 | KLVFFAEDV | 453.270 |
| | | SEQ ID NO. 25 | |
| 2 | 10 | YEVHHQKLV | 6.221 |
| | | SEQ ID NO. 26 | |
| 3 | 4 | FRHDSGYEV | 0.182 |
| | | SEQ ID NO. 27 | |
| 4 | 13 | HHQKLVFFA | 0.009 |
| | | SEQ ID NO. 28 | |
| 5 | 17 | LVFFAEDVG | 0.008 |
| | | SEQ ID NO. 29 | |

b) Sequence
DAEFRHDSGYEVHHQKEEFFAEDVGSNKGA,
SEQ ID NO. 30 (LV/EE modification)

| | | | |
|---|---|---|---|
| 1 | 16 | KEEFFAEDV | 0.564 |
| | | SEQ ID NO. 31 | |
| 2 | 4 | FRHDSGYEV | 0.182 |
| | | SEQ ID NO. 32 | |
| 3 | 2 | AEFRHDSGY | 0.005 |
| | | SEQ ID NO. 33 | |
| 4 | 13 | HHQKEEFFA | 0.004 |
| | | SEQ ID NO. 34 | |
| 5 | 10 | YEVHHQKEE | 0.001 |
| | | SEQ ID NO. 35 | |

c) Sequence
DAEFRHDSGYEVHHQKDDFFAEDVGSNKGA,
SEQ ID NO. 36 (LV/DD modification)

| | | | |
|---|---|---|---|
| 1 | 16 | KDDFFAEDV | 0.252 |
| | | SEQ ID NO. 37 | |
| 2 | 4 | FRHDSGYEV | 0.182 |
| | | SEQ ID NO. 38 | |
| 3 | 2 | AEFRHDSGY | 0.005 |
| | | SEQ ID NO. 39 | |
| 4 | 13 | HHQKDDFFA | 0.004 |
| | | SEQ ID NO. 40 | |
| 5 | 10 | YEVHHQKDD | 0.001 |
| | | SEQ ID NO. 41 | |

d) Sequence
DAEFRHDSGYEVHHQKKKFFAEDVGSNKGA,
SEQ ID No. 42(LV/KK modification)

| | | | |
|---|---|---|---|
| 1 | 4 | FRHDSGYEV | 0.182 |
| | | SEQ ID NO. 42 | |
| 2 | 16 | KKKFFAEDV | 0.022 |
| | | SEQ ID NO. 43 | |
| 3 | 2 | AEFRHDSGY | 0.005 |
| | | SEQ ID NO. 44 | |
| 4 | 13 | HHQKKKFFA | 0.004 |
| | | SEQ ID NO. 45 | |
| 5 | 10 | YEVHHQKKK | 0.001 |
| | | SEQ ID NO. 46 | |

e) Sequence
DAEFRHDSGYEVHHQKEEEFAEDVGSNKGA
SEQ ID NO. 47 (LVF/EEE modification)

| | | | |
|---|---|---|---|
| 1 | 16 | KEEEFAEDV | 2.313 |
| | | SEQ ID NO. 47 | |
| 2 | 4 | FRHDSGYEV | 0.182 |
| | | SEQ ID NO. 48 | |
| 3 | 2 | AEFRHDSGY | 0.005 |
| | | SEQ ID NO. 49 | |
| 4 | 10 | YEVHHQKEE | 0.001 |
| | | SEQ ID NO. 50 | |
| 5 | 8 | SGYEVHHQK | 0.001 |
| | | SEQ ID NO. 51 | |

f) Sequence
DAEFRHDSGYEVHHQKEDDFAEDVGSNKGA,
SEQ ID No. 52, (LVF/EDD modification)

| | | | |
|---|---|---|---|
| 1 | 16 | KEDDFAEDV | 14.454 |
| | | SEQ ID NO. 52 | |
| 2 | 4 | FRHDSGYEV | 0.182 |
| | | SEQ ID NO. 53 | |
| 3 | 2 | AEFRHDSGY | 0.005 |
| | | SEQ ID NO. 54 | |
| 4 | 10 | YEVHHQKED | 0.001 |
| | | SEQ ID NO. 55 | |
| 5 | 8 | SGYEVHHQK | 0.001 |
| | | SEQ ID NO. 56 | |

### Example 3

The binding (or lack thereof) of HLA-A2.01 epitopes derived from the Abeta homologue LV/EE (modifications at positions 17 and 18) was tested in an in vitro system to validate the computer predictions. Comparisons were made to predicted epitopes from the wild-type human Abeta 1-42 sequence. Recombinant HLA A0201 heavy chains were produced in E. coli and purified from inclusion bodies according to a standard procedure described elsewhere (Ostergaard Pederson, L. et al. 2001). Briefly, HLA 0201 heavy chains (1 nM were incubated for 4 hr. at room temperature with 1 nM iodinated control binding peptide (FLPSDYFPSV-SEQ ID No. 1; this peptide has a score of 607.884 when submitted to the ILA Peptide Binding Prediction program at BIMAS), 1000 nM human β2M and graded doses (indicated in figures) of unlabelled peptide of interest (derived from Abeta or its homologue). Receptor bound and free peptide were separated by G25 spun column chromatography (Buus, S. et al. 1995) and counted in a gamma counter (Cobra). The peptides used in this study are listed in Table 4:

TABLE 4

| | | Epitope used | |
|---|---|---|---|
| | | for Binding | |
| Peptide # | Derived from Sequence | Studies | Predicted Score |
| 2 | 1-42 | KLVFFAEDV | 453.270 |
| | | SEQ ID NO. 57 | |
| 1 | 1-42 | GLMVGGVVI | 15.827 |
| | | SEQ ID NO. 58 | |
| 9 | 1-42 | YEVHHQKLV | 6.221 |
| | | SEQ ID NO. 59 | |
| 10 | 1-42 | LMVGGVVIA | 5.752 |
| | | SEQ ID NO. 59 | |
| 6 | 1-42 | IIGLMVGGV | 4.861 |
| | | SEQ ID NO. 60 | |
| 7 | 1-30 LV/EE | KEEFFAEDV | 0.564 |
| | | SEQ ID NO. 61 | |
| 8 | 1-30 LV/EE | FRHDSGYEV | 0.182 |
| | | SEQ ID NO. 62 | |
| 5 | 1-30 LV/EE | AEFRHDSGY | 0.005 |
| | | SEQ ID NO. 63 | |
| 3 | 1-30 LV/EE | HHQKEEFFA | 0.004 |
| | | SEQ ID NO. 64 | |
| 4 | 1-30 LV/EE | YEVHHQKEE | 0.001 |
| | | SEQ ID NO. 65 | |

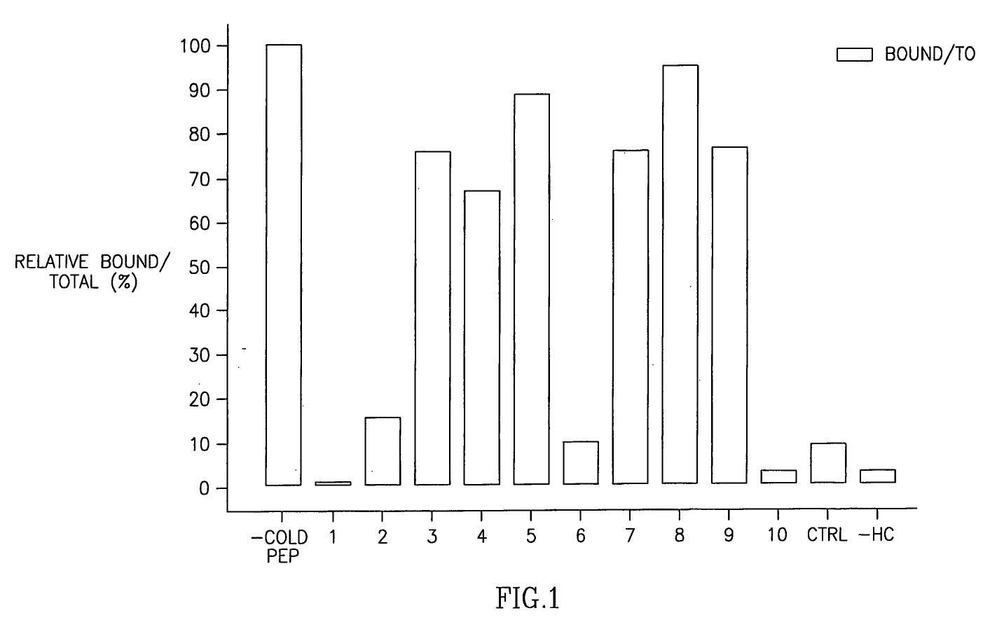

FIG. 1 shows the results of an initial screening of the 10 epitopes for their ability to compete away the binding of the control radiolabeled peptide to recombinant HLA-A201 molecules. Peptides 1, 2, 6, and 10 were all able to compete with the control radiolabeled peptide for binding to HLA-A201. These four peptides (epitopes) are all derived from the wild-type Abeta 1-42 sequence. It is therefore very likely that these peptides will also elicit a CTL response in human HLA-A201 T-lymphocytes (see prophetic examples below). Peptide 9, also derived from Abeta 1-42, did not bind well in this assay, and may therefore not be relevant for the induction of a CTL response. Importantly, all five peptides derived from the LV/EE homologue (peptides 3, 4, 5, 7, 8) did not bind well to the recombinant HLA-A201 molecules, as predicted, and will therefore most likely not induce a CTL response in lymphocytes with this haplotype. Three of these peptides (3, 5, and 8) are also predicted epitopes with low scores from the homologue with modifications at positions 18 and 19 (VF/EE). Homologue VF/EE has also been shown to have a low propensity to form fibrils in vitro and is not toxic to neuroblastoma cells in culture (Sigurdsson, F. et al., personal communication).

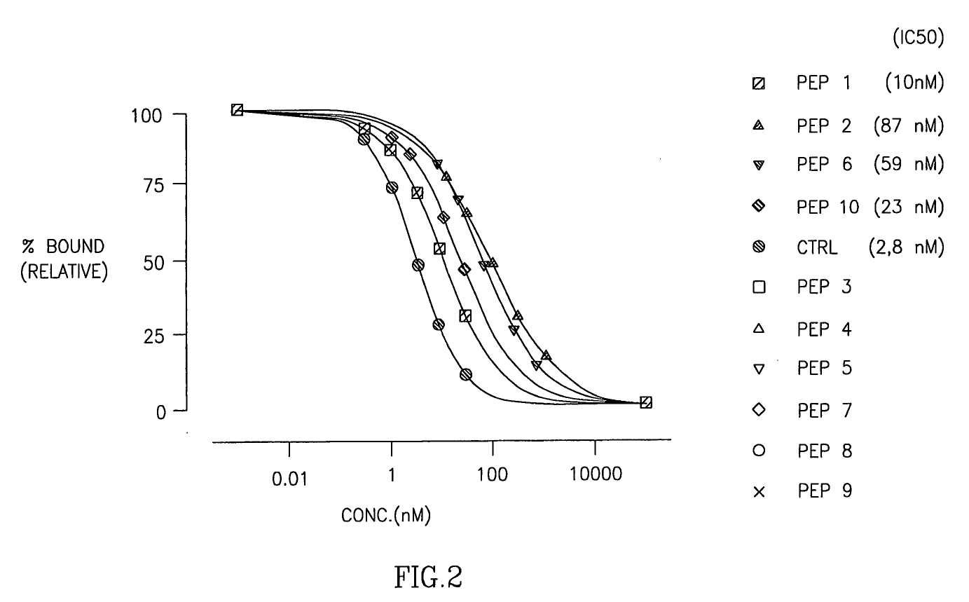

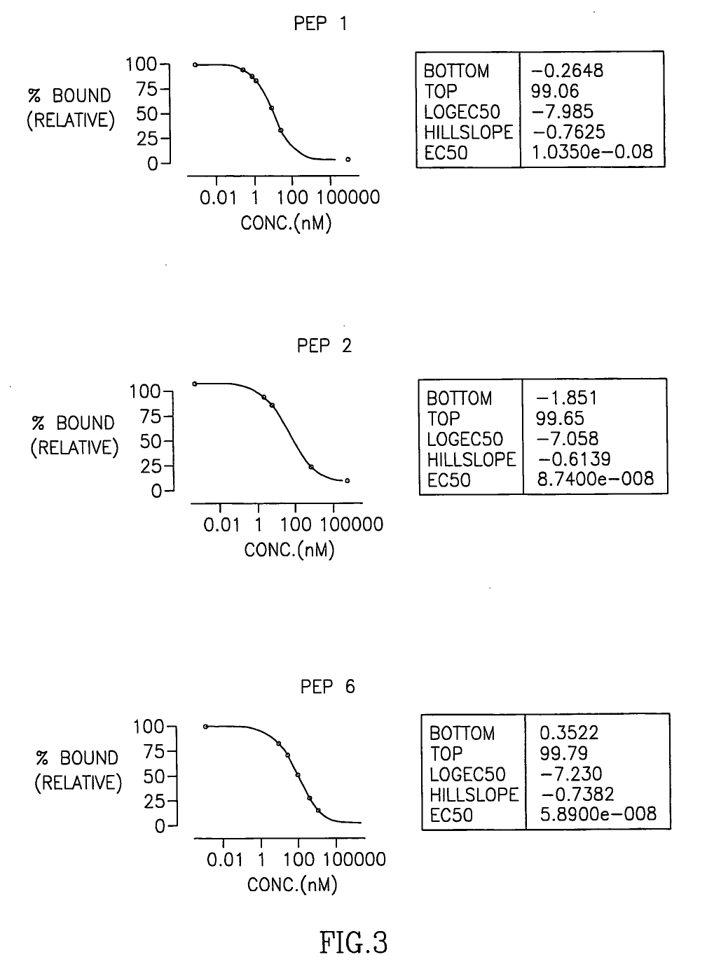

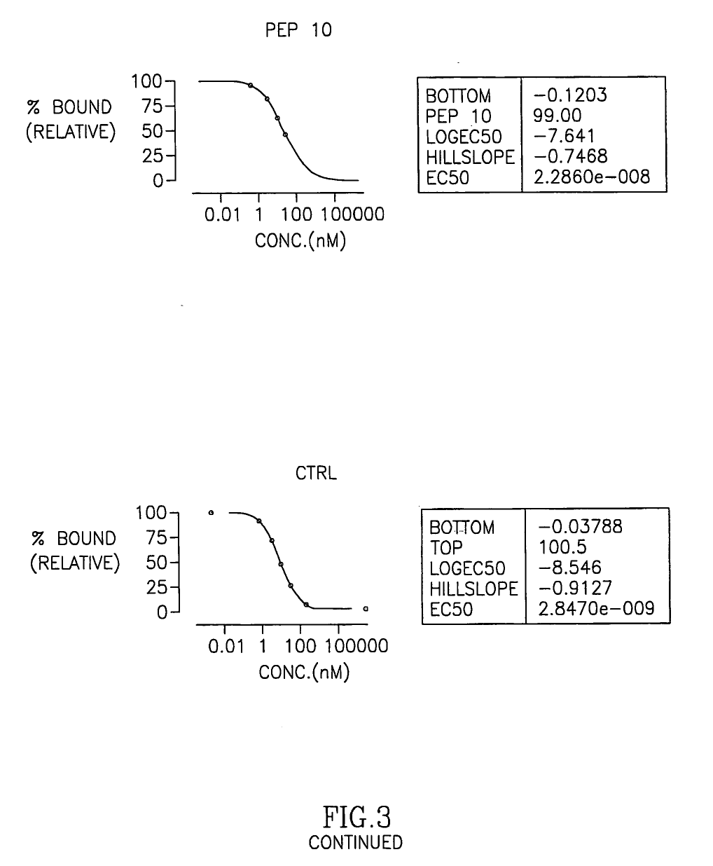

These results were further validated in a secondary screen as depicted in FIGS. 2 and 3. In this experiment, increasing doses of Abeta 1-42 or homologue-derived peptides were used for competition analysis.

### Example 4

According to the allele frequencies of serologically typed HLA loci reported at the XIth Workshop (http://histo.chu-stlouis.fr/inserm/marc/Stats/statser.htm), the four most common HLA-A molecules in the U.S. Caucasian population are A1 (16.9%), A2 (28.3%), A3 (12.2%), and A24 (9.6%). Additional statistics on the frequency of HLA-A, B, and C molecules can be found in the book entitled The HLA Factsbook (Academic Press, 2000).Screening of peptide Abeta K6-1-30-LV/EE for these prevalent alleles gives the results shown in Table 5. No epitopes of significance are predicted to bind to HLA-A2_{—}01, A2_{—}05, or A3 molecules (Table 5c-d). The very low score of the highest ranked epitope for the HLA-A24 molecule (score of 2.2; Table 5e) suggests that this will also not be if significance. The HLA-A1 allele, on the other hand, shows binding to an epitope from the Abeta K6-1-30-LV/EE with a score of 18 (Table 5a). If this epitope is validated in in vitro assays (see below), it would be prohibitive to administer the K6-1-30-LV/EE peptide to individuals displaying the HLA-A1 molecule. It is important to note that the addition of the K6 motif at the N-terminus of Ab1-30 does not introduce any epitopes of significance for the above-mentioned HLA alleles.

TABLE 5
Analysis of peptide predictions based on binding
of subsequences from the Aβhomolog K6-1-30-LV/EE (KK
KKKKDAEFRHDSGYEVHHQKEEFFAEDVGSNKGA,
SEQ ID NO. 66) to prevalent HLA-A molecules in the
Caucasian population (A1, A2, A3, and A24).
Scoring Results

| | | | |
|---|---|---|---|
| | | | Score (Estimate of Half Time of |
| | Start | Subsequence Residue | Disassociation of a Molecule |
| Rank | Position | Listing | Containing This Subsequence) |

a) HLA molecule type selected A1

| | | | |
|---|---|---|---|
| 1 | 26 | FAEDVGSNK | 18.000 |
| | | SEQ ID NO. 67 | |
| 2 | 7 | DAEFRHDSG | 0.900 |
| | | SEQ ID NO. 68 | |
| 3 | 17 | EVHHQKEEF | 0.100 |
| | | SEQ ID NO. 69 | |
| 4 | 14 | SGYEVHHQK | 0.050 |
| | | SEQ ID NO. 70 | |
| 5 | 22 | KEEFFAEDV | 0.045 |
| | | SEQ ID NO. 71 | |

b) HLA molecule type selected A_0201

| | | | |
|---|---|---|---|
| 1 | 22 | KEEFFAEDV | 0.564 |
| | | SEQ ID NO. 72 | |
| 2 | 10 | FRHDSGYEV | 0.182 |
| | | SEQ ID NO. 73 | |
| 3 | 6 | KDAEFRHDS | 0.006 |
| | | SEQ ID NO. 74 | |
| 4 | 8 | AEFRHDSGY | 0.005 |
| | | SEQ ID NO. 75 | |
| 5 | 19 | HHQKEEFFA | 0.004 |
| | | SEQ ID NO. 76 | |

c) HLA molecule type selected A_0205

| | | | |
|---|---|---|---|
| 1 | 22 | KEEFAEDV | 0.336 |
| | | SEQ ID NO. 77 | |
| 2 | 10 | FRHDSGYEV | 0.018 |
| | | SEQ ID NO. 78 | |
| 3 | 8 | AEFRHDSGY | 0.003 |
| | | SEQ ID NO. 79 | |
| 4 | 19 | HHQKEEFFA | 0.003 |
| | | SEQ ID NO. 80 | |
| 5 | 6 | KDAEFRHDS | 0.001 |
| | | SEQ ID NO. 81 | |

d) HLA molecule type selected A3

| | | | |
|---|---|---|---|
| 1 | 26 | FAEDVGSNK | 0.300 |
| | | SEQ ID NO. 82 | |
| 2 | 14 | SGYEVHHQK | 0.225 |
| | | SEQ ID NO. 83 | |
| 3 | 17 | EVHHQKEEF | 0.060 |
| | | SEQ ID NO. 84 | |
| 4 | 8 | AEFRHDSGY | 0.060 |
| | | SEQ ID NO. 85 | |
| 5 | 3 | KKKKDAEFR | 0.012 |
| | | SEQ ID NO. 86 | |

e) HLA molecule type selected A24

| | | | |
|---|---|---|---|
| 1 | 17 | EVHHQKEEF | 2.200 |
| | | SEQ ID NO. 87 | |
| 2 | 15 | GYEVHHQKE | 0.990 |
| | | SEQ ID NO. 88 | |
| 3 | 25 | FFAEDVGSN | 0.600 |
| | | SEQ ID NO. 89 | |
| 4 | 24 | EFFAEDVGS | 0.500 |
| | | SEQ ID NO. 90 | |
| 5 | 2 | KKKKKDAEF | 0.440 |
| | | SEQ ID NO. 91 | |

### Example 5

According to the allele frequencies of serologically typed HLA loci reported at the XIth Workshop (http://histo.chu-stlouis.fr/inserm/marc/Stats/statser.htm), the most common HLA-B molecules in the Japanese (Wajin) population are B52, B61, B51, B62, and B35. Screening of peptide Abeta K6-1-30-LV/EE for these prevalent alleles gives the results shown in Table 5. No epitopes of significance are predicted to bind to HLA-10 B_{—}5201, B_{—}5101, B_{—}5102, B_{—}5103, B62, or B_{—}3501 molecules (Table 6a, c-g). The BLA-B61 allele, on the other hand, shows binding to an epitope from the Abeta K6-1-30-LV/EE with a score of 40 (Table 6b). If this epitope is validated in in vitro assays (see below), it would be prohibitive to administer the K6-1-30-LV/EE peptide to individuals displaying the HLA-B61 molecule. It is important to note that the addition of the K6 motif at the N-terminus of Ab1-30 does not introduce any epitopes of significance for the above-mentioned HLA alleles.

TABLE 6
Analysis of peptide predictions based on binding
of subsequences from the Aβhomolog K6-1-30-LV/EE (KK
KKKKDAEFRHDSGYEVHHQKEEFFAEDVGSNKGA,
SEQ ID No. 92) to prevalent HLA-B molecules in the
Japanese (Wajin) population (B52, B61, B51, B62, and B35.
Scoring Results

| | | | |
|---|---|---|---|
| | | | Score (Estimate of Half Time of |
| | Start | Subsequence Residue | Disassociation of a Molecule |
| Rank | Position | Listing | Containing This Subsequence) |

a) HLA molecule type selectedB_5201

| | | | |
|---|---|---|---|
| 1 | 22 | KEEFFAEDV | 1.650 |
| | | SEQ ID NO. 93 | |
| 2 | 23 | EEFFAEDVG | 0.750 |
| | | SEQ ID NO. 94 | |
| 3 | 17 | EVHHQKEEF | 0.605 |
| | | SEQ ID NO. 95 | |
| 4 | 14 | SGYEVHHQK | 0.600 |
| | | SEQ ID NO. 96 | |
| 5 | 8 | AEFRHDSGY | 0.500 |
| | | SEQ ID NO. 97 | |

b) HLA molecule type selected B61

| | | | |
|---|---|---|---|
| 1 | 22 | KEEFFAEDV | 40.000 |
| | | SEQ ID NO. 98 | |
| 2 | 28 | EDVGSNKGA | 5.000 |
| | | SEQ ID NO. 99 | |
| 3 | 8 | AEFRHDSGY | 2.400 |
| | | SEQ ID NO. 100 | |
| 4 | 23 | EEFFAEDVG | 1.200 |
| | | SEQ ID NO. 101 | |
| 5 | 16 | YEVHHQKEE | 0.800 |
| | | SEQ ID NO. 102 | |

c) HLA molecule type selected B_5101

| | | | |
|---|---|---|---|
| 1 | 7 | DAEFRHDSG | 1.000 |
| | | SEQ ID NO. 103 | |
| 2 | 26 | FAEDVGSNK | 0.787 |
| | | SEQ ID NO. 104 | |
| 3 | 10 | FRHDSGYEV | 0.629 |
| | | SEQ ID NO. 105 | |
| 4 | 14 | SGYEVHHQK | 0.484 |
| | | SEQ ID NO. 106 | |
| 5 | 22 | KEEFFAEDV | 0.220 |
| | | SEQ ID NO. 107 | |

d) HLA molecule type selected B_5102

| | | | |
|---|---|---|---|
| 1 | 14 | SGYEVHHQK | 1.210 |
| | | SEQ ID NO. 108 | |
| 2 | 10 | FRHDSGYEV | 0.800 |
| | | SEQ ID NO. 109 | |
| 3 | 26 | FAEDVGSNK | 0.550 |
| | | SEQ ID NO. 110 | |
| 4 | 24 | EFFAEDVGS | 0.250 |
| | | SEQ ID NO. 111 | |
| 5 | 7 | DAEFRHDSG | 0.250 |
| | | SEQ ID NO. 112 | |

e) HLA molecule type selected B_5103

| | | | |
|---|---|---|---|
| 1 | 14 | SGYEVHHQK | 0.726 |
| | | SEQ ID NO. 113 | |
| 2 | 7 | DAEFRHDSG | 0.605 |
| | | SEQ ID NO. 114 | |
| 3 | 26 | FAEDVGSNK | 0.550 |
| | | SEQ ID NO. 115 | |
| 4 | 10 | FRHDSGYEV | 0.400 |
| | | SEQ ID NO. 116 | |
| 5 | 22 | KEEFFAEDV | 0.400 |
| | | SEQ ID NO. 117 | |

f) HLA molecule type selected B62

| | | | |
|---|---|---|---|
| 1 | 20 | HQKEEFFAE | 1.320 |
| | | SEQ ID NO. 118 | |
| 2 | 17 | EVHHQKEEF | 1.000 |
| | | SEQ ID NO. 119 | |
| 3 | 2 | KKKKKDAEF | 0.300 |
| | | SEQ ID NO. 120 | |
| 4 | 18 | VHHQKEEFF | 0.100 |
| | | SEQ ID NO. 121 | |
| 5 | 8 | AEFRHDSGY | 0.100 |
| | | SEQ ID NO. 122 | |

g) HLA molecule type selected B_3501

| | | | |
|---|---|---|---|
| 1 | 17 | EVHHQKEEF | 1.000 |
| | | SEQ ID NO. 123 | |
| 2 | 2 | KKKKKDAEF | 0.600 |
| | | SEQ ID NO. 124 | |
| 3 | 8 | AEFRHDSGY | 0.200 |
| | | SEQ ID NO. 125 | |
| 4 | 18 | VHHQKEEFF | 0.100 |
| | | SEQ ID NO. 126 | |
| 5 | 20 | HQKEEFFAE | 0.090 |
| | | SEQ ID NO. 127 | |

### Example 6

The sequence of Ab1-42 and the sequence of 1-30VF/EE i.e the 1-30 amyloid beta peptide, wherein the VF was replaced by EE, were entered into the algorithm RANKPEP (Reche P A et al. 2002). This program ranks all possible peptides from an input protein sequence/s by their similarity to a set of peptides known to bind to a given MHC molecule. Similarity is scored using a Position Specific Scoring Matrix (PSSM) built from a collection of aligned peptides known to bind to that MHC molecule. Using the subsequence length of 15 amino acids, analysis was done for the following HLA Class II options: HLA_DRB1_{—}0101 (HLA-DR1), HLA_DRB1_{—}1501 (HLA-DR2b), HLA_DRB5_{—}0101 (HLA-DR2a), HLA_DRB1_{—}03 (HLA-DR3), HLA_DRB1_{—}0401 (HLA-DR4), HLA_DQA1_{—}0301_DQB1_{—}0302 (BLA-DQ8).

The results can be classified into three categories: a) epitopes which do not exist in the K61-30VF/EE peptide because they require residues between amino acids 31-42. b) epitopes that have a reduced score or are eliminated due to the internal modifications of EE at positions 18 and 19; c) epitopes that have an increased score or are added as a result of the internal modifications of EE at positions 18 and 19. Tables 7 and 8 are exemplary of this type of analysis, performed on the seven prevalent HLA class II molecules. No significant changes in the general outcome (number of binders) were predicted for the alleles HLA_DRB1_{—}0101, HLA_DRB5_{—}0101, and HLA_DRB1_{—}03. For allele HLA_DRB1-1501, the Abeta homologue has only one predicted binding epitope, compared to two in the Abeta 1-42 sequence. The VF to EE modification has eliminated an important binding epitope. A similar situation is seen for allele HLA-DRB1_{—}0401, in which two binding epitopes are eliminated in the K61-30 VF/EE homologue. It seems that the opposite result occurs with allele HLA_DQA1_{—}0301_DQB1_{—}0302, in which five binding epitopes appear in the K61-30 VF/EE homologue as opposed to the Abeta 1-42 sequence. However, three of these new epitopes include large parts of the K6 N-terminal tail and therefore are not expected to initiate an immune response to Abeta sequences per se. In fact, this K6 tail was chosen for its ability to be immunogenic and this may be part of the expected T-helper response.

A systematic analysis can be performed in the above manner for choice of antigenic peptide that will not induce harmful T-cell autoimmunity in a large population of vaccine patients. Alternatively, a number of vaccine antigens can be developed and chosen on an individual basis for administration according to HLA haplotype. In either case a method of screening vaccine candidates is essential in order to determine their haplotype and either their suitability for a certain vaccine antigen or to chose from a pool of antigens that which would be best matched to them.

TABLE 7
HLA Class II binding predictions for Abeta 1-42

| | | | | | | | |
|---|---|---|---|---|---|---|---|
| RANK | POS | N | SEQUENCE | C | MW (Da) | SCORE | % OPT. |

a)
Matrix: HLA_DRB1_0101.pwp
Consensus: YKAMRAAAA
Optimal Score: 133.0
Binding Threshold: 14.00

| | | | | | | | |
|---|---|---|---|---|---|---|---|
| 1 | 10 | DSG | YEVHHQKLV | FFA | 1134.3 | 50.0 | 37.59% |
| | | | SEQ ID NO. 128 | | | | |
| 2 | 4 | DAE | FRHDSGYEV | HHQ | 1091.16 | 44.0 | 33.08% |
| | | | SEQ ID NO. 129 | | | | |
| 3 | 20 | LVF | FAEDVGSNK | GAI | 948.0 | 39.0 | 29.32% |
| | | | SEQ ID NO. 130 | | | | |
| 4 | 24 | AED | VGSNKGAII | GLM | 839.98 | 36.0 | 27.07% |
| | | | SEQ ID NO. 131 | | | | |
| 5 | 32 | GAI | IGLMVGGVV | IA | 826.05 | 34.0 | 25.56% |
| | | | SEQ ID NO. 132 | | | | |

b)
Matrix: HLA_DRB1_1501.pwp
Consensus: VHFAKNTAT
Optimal Score: 130.0
Binding Threshold: 49.00

| | | | | | | | |
|---|---|---|---|---|---|---|---|
| 2 | 17 | HQK | LVFFAEDVG | SNK | 978.12 | 51.0 | 39.23% |
| | | | SEQ ID NO. 134 | | | | |
| 3 | 12 | GYE | VHHQKLVFF | AED | 1136.36 | 34.0 | 26.15% |
| | | | SEQ ID NO. 135 | | | | |
| 4 | 2 | D | AEFRHDSGY | EVH | 1063.11 | 31.0 | 23.85% |
| | | | SEQ ID NO. 136 | | | | |
| 5 | 20 | LVF | FAEDVGSNK | GAI | 948.0 | 23.0 | 17.69% |
| | | | SEQ ID NO. 137 | | | | |

c)
Matrix: HLA_DRB5_0101.pwp
Consensus: YAAAKAAAK
Optimal Score: 149.0
Binding Threshold: 60.00

| | | | | | | | |
|---|---|---|---|---|---|---|---|
| 1 | 10 | DSG | YEVHHQKLV | FFA | 1134.3 | 53.0 | 35.57% |
| | | | SEQ ID NO. 138 | | | | |
| 2 | 20 | LVF | FAEDVGSNK | GAI | 948.0 | 43.0 | 28.86% |
| | | | SEQ ID NO. 139 | | | | |
| 3 | 19 | KLV | FFAEDVGSN | KGA | 967.01 | 36.0 | 24.16% |
| | | | SEQ ID NO. 140 | | | | |
| 4 | 4 | DAE | FRHDSGYEV | HHQ | 1091.16 | 34.0 | 22.82% |
| | | | SEQ ID NO. 141 | | | | |
| 5 | 24 | AED | VGSNKGAII | GLM | 839.98 | 33.0 | 22.15% |
| | | | SEQ ID NO. 142 | | | | |

d)
Matrix: HLA_DRB1_03.pwp
Consensus: LSLDTESRY
Optimal Score: 164.0
Binding Threshold: 74.00

| | | | | | | | |
|---|---|---|---|---|---|---|---|
| 1 | 20 | LVF | FAEDVGSNK | GAI | 948.0 | 60.0 | 36.59% |
| | | | SEQ ID NO. 143 | | | | |
| 2 | 32 | GAI | IGLMVGGVV | IA | 826.05 | 52.0 | 31.71% |
| | | | SEQ ID NO. 144 | | | | |
| 3 | 10 | DSG | YEVHHQKLV | FFA | 1134.3 | 46.0 | 28.05% |
| | | | SEQ ID NO. 145 | | | | |
| 4 | 4 | DAE | FRHDSGYEV | HHQ | 1091.16 | 45.0 | 27.44% |
| | | | SEQ ID NO. 146 | | | | |
| 5 | 8 | RHD | SGYEVHHQK | LVF | 1066.14 | 32.0 | 19.51% |
| | | | SEQ ID NO. 147 | | | | |

e)
Matrix: HLA_DRB1_0401.pwp
Consensus: YASSSTMSA
Optimal Score: 107.0
Binding Threshold: 22.00

| | | | | | | | |
|---|---|---|---|---|---|---|---|
| 1 | 20 | LVF | FAEDVGSNK | GAI | 948.0 | 42.0 | 39.25% |
| | | | SEQ ID NO. 148 | | | | |
| 2 | 4 | DAE | FRHDSGYEV | HHQ | 1091.16 | 37.0 | 34.58% |
| | | | SEQ ID NO. 149 | | | | |
| 3 | 10 | DSG | YEVHHQKLV | FFA | 1134.3 | 31.0 | 28.97% |
| | | | SEQ ID NO. 150 | | | | |
| 4 | 19 | KLV | FFAEDVGSN | KGA | 967.01 | 29.0 | 27.10% |
| | | | SEQ ID NO. 151 | | | | |
| 5 | 34 | IIG | LMVGGVVIA | | 840.08 | 26.0 | 24.30% |
| | | | SEQ ID NO. 152 | | | | |

f)
Matrix: HLA_DQA1_0301_DQB1_0302.pwp
Consensus: DMRSFPEVK
Optimal Score: 125.0
Binding Threshold: 45.00

| | | | | | | | |
|---|---|---|---|---|---|---|---|
| 1 | 3 | DA | EFRHDSGYE | VHH | 1121.15 | 44.0 | 35.20% |
| | | | SEQ ID NO.153 | | | | |
| 2 | 5 | AEF | RHDSGYEVH | HQK | 1081.12 | 39.0 | 31.20% |
| | | | SEQ ID NO. 154 | | | | |
| 3 | 23 | FAE | DVGSNKGAI | IGL | 841.91 | 36.0 | 28.80% |
| | | | SEQ ID NO. 155 | | | | |
| 4 | 11 | SGY | EVHHQKLVF | FAE | 1118.3 | 31.0 | 24.80% |
| | | | SEQ ID NO. 156 | | | | |
| 5 | 16 | HHQ | KLVFFAEDV | GSN | 1049.24 | 21.0 | 16.80% |
| | | | SEQ ID NO. 157 | | | | |

TABLE 8
HLA Class II binding predictions for K6 1-30 VF/EE

| | | | | | | | |
|---|---|---|---|---|---|---|---|
| RANK | POS. | N | SEQUENCE | C | MW (Da) | SCORE | % OPT. |

a)
Matrix: HLA_DRB1_0101.pwp
Consensus: YKAMRAAAA
Optimal Score: 133.0
Binding Threshold: 14.00

| | | | | | | | |
|---|---|---|---|---|---|---|---|
| 1 | 4 | DAE | FRHDSGYEV | HHQ | 1091.16 | 44.0 | 33.08% |
| | | | SEQ ID NO. 158 | | | | |
| 2 | 14 | EVH | HQKLEEFAE | DVG | 1112.22 | 40.0 | 30.08% |
| | | | SEQ ID NO. 159 | | | | |
| 3 | 20 | LEE | FAEDVGSNK | GA | 948.0 | 39.0 | 29.32% |
| | | | SEQ ID NO. 160 | | | | |
| 4 | 10 | DSG | YEVHHQKLE | EFA | 1164.29 | 39.0 | 29.32% |
| | | | SEQ ID NO. 161 | | | | |
| 5 | 17 | HQK | LEEFAEDVG | SNK | 990.05 | 18.0 | 13.53% |
| | | | SEQ ID NO. 162 | | | | |

b)
Matrix: HLA_DRB1_1501.pwp
Consensus: VHFAKNTAT
Optimal Score: 130.0
Binding Threshold: 49.00

| | | | | | | | |
|---|---|---|---|---|---|---|---|
| 1 | 24 | QKL | EEFAEDVGS | NKG | 963.97 | 52.0 | 40.00% |
| | | | SEQ ID NO. 163 | | | | |
| 2 | 23 | HQK | LEEFAEDVG | SNK | 990.05 | 31.0 | 23.85% |
| | | | SEQ ID NO. 164 | | | | |
| 3 | 18 | GYE | VHHQKLEEF | AED | 1148.29 | 31.0 | 23.85% |
| | | | SEQ ID NO. 165 | | | | |
| 4 | 8 | KKD | AEFRHDSGY | EVH | 1063.11 | 31.0 | 23.85% |
| | | | SEQ ID NO. 166 | | | | |
| 5 | 19 | YEV | HHQKLEEFA | EDV | 1120.24 | 25.0 | 19.23% |
| | | | SEQ ID NO. 167 | | | | |

c)
Matrix: HLA_DRB5_0101.pwp
Consensus: YAAAKAAAK
Optimal Score: 149.0
Binding Threshold: 60.00

| | | | | | | | |
|---|---|---|---|---|---|---|---|
| 1 | 16 | DSG | YEVHHQKLE | EFA | 1164.29 | 57.0 | 38.26% |
| | | | SEQ ID NO. 168 | | | | |
| 2 | 20 | EVH | HQKLEEFAE | DVG | 1112.22 | 55.0 | 36.91% |
| | | | SEQ ID NO. 169 | | | | |
| 3 | 26 | LEE | FAEDVGSNK | GA | 948.0 | 43.0 | 28.86% |
| | | | SEQ ID NO. 170 | | | | |
| 4 | 3 | KK | KKKKDAEFR | HDS | 1131.34 | 37.0 | 24.83% |
| | | | SEQ ID NO. 171 | | | | |
| 5 | 10 | DAE | FRHDSGYEV | HHQ | 1091.16 | 34.0 | 22.82% |
| | | | SEQ ID NO. 172 | | | | |

d)
Matrix: HLA_DRB1_03.pwp
Consensus: LSLDTESRY
Optimal Score: 164.0
Binding Threshold: 74.00

| | | | | | | | |
|---|---|---|---|---|---|---|---|
| 1 | 26 | LEE | FAEDVGSNK | GA | 948.0 | 60.0 | 36.59% |
| | | | SEQ ID NO. 173 | | | | |
| 2 | 4 | KKK | KKKDAEFRH | DSG | 1140.31 | 54.0 | 32.93% |
| | | | SEQ ID NO. 174 | | | | |
| 3 | 10 | DAE | FRHDSGYEV | HHQ | 1091.16 | 45.0 | 27.44% |
| | | | SEQ ID NO. 175 | | | | |
| 4 | 16 | DSG | YEVHHQKLE | EFA | 1164.29 | 38.0 | 23.17% |
| | | | SEQ ID NO. 176 | | | | |
| 5 | 21 | VHH | QKLEEFAED | VGS | 1090.17 | 36.0 | 21.95% |
| | | | SEQ ID NO. 177 | | | | |

e)
Matrix: HLA_DRB1_0401.pwp
Consensus: YASSSTMSA
Optimal Score: 107.0
Binding Threshold: 22.00

| | | | | | | | |
|---|---|---|---|---|---|---|---|
| 1 | 26 | LEE | FAEDVGSNK | GA | 948.0 | 42.0 | 39.25% |
| | | | SEQ ID NO. 178 | | | | |
| 2 | 10 | DAE | FERHDSGYEV | HHQ | 1091.16 | 37.0 | 34.58% |
| | | | SEQ ID NO. 179 | | | | |
| 3 | 16 | DSG | YEVHHQKLE | EFA | 1164.29 | 32.0 | 29.91% |
| | | | SEQ ID NO. 180 | | | | |
| 4 | 20 | EVH | HQKLEEFAE | DVG | 1112.22 | 16.0 | 14.95% |
| | | | SEQ ID NO. 181 | | | | |
| 5 | 23 | HQK | LEEFAEDVG | SNK | 990.05 | 9.0 | 8.41% |
| | | | SEQ ID NO. 182 | | | | |

f)
Matrix: HLA_DQA1_0301_DQB1_0302.pwp
Consenus: DMRSFPEVK
Optimal Score: 125.0
Binding Threshold: 45.00

| | | | | | | | |
|---|---|---|---|---|---|---|---|
| 1 | 1 | | KKKKKKDAE | FRH | 1084.31 | 79.0 | 63.20% |
| | | | SEQ ID NO. 183 | | | | |
| 2 | 3 | KK | KKKKDAEFR | HDS | 1131.34 | 71.0 | 56.80% |
| | | | SEQ ID NO. 184 | | | | |
| 3 | 22 | HHQ | KLEEFAEDV | GSN | 1061.17 | 60.0 | 48.00% |
| | | | SEQ ID NO. 185 | | | | |
| 4 | 19 | YEV | HHQKLEEFA | EDV | 1120.24 | 51.0 | 40.80% |
| | | | SEQ ID NO. 186 | | | | |
| 5 | 2 | K | KKKKKDAEF | RHD | 1103.32 | 47.0 | 37.60% |
| | | | SEQ ID NO. 187 | | | | |

### PROPHETIC EXAMPLES

### In Vitro Assays for T-Cell Responses

Other important factors include the ability of the cellular antigen processing machinery to generate a certain peptide-MHC complex and the presence or absence of circulating T-cells which can recognize this complex. Many molecules have been identified that participate in the process of antigen presentation including the proteasome, a multicatalytic protease and TAP (transporters associated with antigen processing) molecules, both of which appear to have peptide-dependent activity that is biased to certain amino acid residues and sequences. During the course of development, the fate of immature lymphocytes will be determined by the specificity of its antigen receptor. T-cell precursors with strongly self-reactive receptors will be eliminated to prevent autoimmune reactions; this negative selection allows for self-tolerance of an individual. Also, a process of positive selection identifies and preserves only those T-cell precursors which are likely to respond to foreign antigens. Those that do not pass this test, usually because of very low affinity of T-cell receptor to peptide/MHC complex, will die by neglect. Thus, the peptide binding forecast obtained from predictive programs are only a starting point for determination of important T-cell epitopes. Antigen processing events and T-cell survival clearly influence the reality of these predictions. Thus it is important to validate that the Abeta peptide homologs with binding epitopes removed do not in fact elicit T-cell responses in humans. Some assays to test T-cell responses after in vitro stimulation include: cytotoxicity assays, proliferation assays, cytokine measurements, flow cytometry analyses.

Isolation and growth of T-cells: Human peripheral blood mononuclear cells are separated from diluted anticoagulated blood using Ficoll-Hypaque density gradient separation. The interface includes mononuclear cells which are washed free of residual Ficoll and grown in culture typically using RPMI, 10% human AB serum, specific cytokines such as IL-2, and 5-100 μM peptide. Peptide is typically first pulsed onto adherent antigen presenting cells with P-2-microglobulin. Alternatively, dendritic cells from the same donor can be generated with GM-CSF and EL-4 prior to stimulation and used as antigen presenting cells. Also, donor lymphocytes can be enriched for CD8+ (cytotoxic) or CD4+ (helper) cells, before or after peptide stimulation, using standard techniques, such as positive selection with anti-CD8 or anti-CD4 columns or magnetic beads, panning of cells over antibody-coated plastic surfaces, or passing cells over columns of antibody-coated nylon-coated steel wool. Lymphocytes are restimulated usually once or twice a week with autologous PBMC's that have been irradiated and pulsed with the stimulated peptide. After several rounds of stimulation, and when a significant number of peptide-specific cells have been generated, in vitro assays of T-cell responses can be initiated. These can include, but are not limited to cytoxicity assays, proliferation assays, cytokine assays, FACS analyses, limiting dilution, ELISPOT.

Cytotoxicity assay: Activated CD8 T cells generally kill any cells that display the specific peptide:MHC complex they recognize. Target cells are radiolabeled with ⁵¹Cr or ³⁵M and plated together with peptide-specific T-cells at various effector:target ratios. Typical ratios are 100:1, 50:1, 25:1, and 12.5:1. Cells are incubated together for 4-16 hours and culture medium is collected for measurement of radioactive label that has been released from lysed cells. Radiolabeled cells incubated for the same period of time without T-cell cultures give represent background release of radioactive label.

Proliferation assay (3HTdR incorporation into DNA): Target cells are irradiated and incubated together with peptide-specific T-cells at various effector:target ratios. At certain time points, ³H thymidine is added to the culture and after overnight growth, cells are lysed and the radioactivity is measured as an indication of the amount of proliferation of the T-cell population.

Cytokine release assays: One method to measure the responses of T-cell populations is a variant of the antigen-capture ELISA method, called the ELISPOT assay. In this assay, cytokine secreted by individual activated T cells is immobilized as discrete spots on a plastic plate via anti-cytokine antibodies, which are counted to give the number of activated T cells. Another method is to collect culture supernatant from stimulated cells and measure cytokines directly by standard ELISA methods. To test the cytokine profile produced by individual cells, intracellular cytokine staining relies on the use of metabolic poisons to inhibit protein export from the cell. The cytokine thus accumulates within the endoplasmic reticulum and vesicular network of the cells. Once cells are fixed and permeabilized, antibodies can gain access to the intracellular compartments to detect cytokine, using flow cytometry.

Flow cytometry: The activation state of in vitro peptide-stimulated T-cells can be assessed using fluorescence-activated cell sorter or FACS. Cells are washed free of culture medium and incubated with isotype control or specific anti-CD antibody for 1 hr. at 4° C. Either the first antibody or a secondary antibody is labeled with a fluorescent marker. After washing cells free of unbound antibody, they are collected and analyzed by a FACS machine. The percentage of positive cells or the intensity of the fluorescence can give an indication of the activation state of the cells. For examples, markers of T-cell activation include CD69 and CD25, the IL-2 receptor alpha chain. In addition, flow cytometry can be used to detect fluorescently labeled cytokines within activated T cells or the directly detect T cells on the basis of the specificity of their receptor, using fluorochrome-tagged tetramers of specific MHC:peptide complexes.

Additional In Vitro and In Vivo Assays for Peptide Selection:

Antibody production: Abeta peptides or homologues selected for their reduced number or potency of T-cell epitopes must retain the ability to mount an antibody response which will target the Abeta peptide. Standard algorithms and programs which predict antigenicity of peptides and proteins can assist in this regard. Peptides can also be administered in adjuvant to wild-type or preferably to APP transgenic mice or guinea pigs over several weeks or months. Animals are bled periodically and antibody titers to the toxic peptides Abeta 1-40 and 1-42 are tested in standard ELISA, immunoprecipitation, or immunohistochemistry experiments.

### Secondary Structure Studies

Secondary structure (α-helix, β-sheet, and random coil) of the peptides can be evaluated by circular dichroism (CD) as described previously (Soto et al., 1998 and Soto et al., 1996). Results are expressed as molar ellipticity in units of deg cm² dmol⁻¹, and the data was analyzed by the Lincomb and CCA algorithms (Perczel et al., 1992) to obtain the percentages of different types of secondary structure.

Secondary structure of the synthesized peptides can also be evaluated by Fourier-Transform InfraRed spectroscopy (FTIR), using published protocols from Aucouturier et al. (1999). Although CD is sensitive to backbone conformation and FTIR is sensitive to the degree and strength of hydrogen bonding of amide groups (which is dependent of the structure), these two techniques ultimately give similar information: the percentages of different secondary structure motifs, i.e., α-helix, β-sheet, β-turn and random coil (Surewicz et al., 1993). CD is a very well-established technique for studying the secondary structure of proteins and peptides in solution, giving fairly accurate estimations of the content of different structural motifs. A major advantage of FTIR spectroscopy for structural characterization is the lack of dependence on the physical state of the sample. Samples may be examined as aqueous or organic solutions, hydrated films, inhomogeneous dispersions, aggregated materials or even proteins in solid state. Therefore, CD and FTIR are complementary for studying the secondary structure of peptides.

The experimental procedure for circular dichroism is performed according to Golabek et al., (1996) and Soto et al. (1996 and 1998) as follows: CD spectra of solutions containing synthetic peptides (1-5 μM in 300 μl of 10 mM sodium phosphate, pH 7.2) is recorded in a Jasco J-720 spectropolarimeter at 25° C. using a 0.1 cm path-length cell with double distilled, deionized water and TFE (spectroscopy grade) being used as solvents. Calibration of the instrument is performed with an aqueous solution of d-(+)-10-camphorsulfonic acid. Spectra is recorded at 1 nm intervals over the wavelength range 180 to 260 nm and buffer spectra obtained under identical conditions is subtracted.

The experimental procedure for Fourier-Transform InfraRed Spectroscopy according to Aucouturier et al. (1999) is as follows: Solutions or suspensions containing soluble or aggregated synthetic peptides (5-10 mg/ml) will be prepared in H₂O and D₂O buffers at neutral pH, and 10 μl will be loaded into an infrared cell with CaF₂ plates and 6 μm path-length spacer. Spectra will be recorded with a Perkin Elmer model 2000 FTIP spectrophotometer at 25° C., as described (Aucouturier et al., 1999; Soto et al., 1995). For each spectrum, 1000 scans will be collected in the single-beam mode with 2 cm⁻resolution and a 1 cm⁻¹ interval from 4000 to 1000 cm⁻. Smoothing and Fourier self-deconvolution will be applied to increase the spectral resolution in the amide I region (1700-1600 cm⁻¹) and the iterative fitting to Lorentzian line shapes will be carried out to estimate the proportion of each secondary structural element.

### Studies of Amyloid Fibril Formation In Vitro

Studies of amyloid fibril formation in vitro can be performed using published protocols (Castaño et al., 1995; Wisniewski et al., 1991; Wisniewski et al., 1993 and Wisniewski et al., 1994). Aliquots of the synthetic peptides at a concentration ranging between 25-250 μM, prepared in 0.1M Tris, pH 7.4, can be incubated for different times, and their fibril formation compared to that of Aβ1-40 and Aβ1-42. In vitro fibrillogenesis is evaluated by a fluorometric assay based on the fluorescence emission by thioflavine T, as previously described (Soto et al., 1998 and Jameson et al., 1998). Thioflavine T binds specifically to amyloid and this binding procedures a shift in its emission spectrum and a fluorescent enhancement proportional to the amount of amyloid formed (LeVine et al. 1993).

In vitro fibrillogenesis can also be evaluated by three other different methods: (A) A spectrophotometric assay based on the specific interaction of Congo red with amyloid fibrils. After the incubation period, 2 μl of Congo red (1.5 mg/ml) will be added to each sample and incubated in the dark for 1 h. The samples will then be centrifuged at 15,000 rpm for 10 min and the absorbance of the supernatant measured at 490 nm. The amount of amyloid formed is directly proportional to the decrease in the supernatant absorbance (Castaño et al., 1986). (B) A sedimentation assay will be used as described (Soto et al., 1995). Briefly, samples will be centrifuged at 15,000 rpm for 10 min to separate the soluble and aggregated peptide. The amount of material in solution will be analyzed by microbore HPLC using a reverse phase Vydac C4 column and a linear gradient of 3-70% acetonitrile. The percentage of aggregated peptide will be estimated by comparing the area of the peak corresponding to the soluble peptide in each incubated sample with an identical control of non-incubated sample. (C) Additional characterization of fibrillogenesis will be performed by Congo red staining and electron microscopic examination after negative staining (Castaño et al., 1995; Wisniewsi et al., 1991; Wisniewski et al., 1993 and Wisniewski et al., 1994). For electron microscopy, the incubated samples of peptides will be placed on carbon formar-coated 300-mesh nickel grids and stained for 60 seconds with 2% uranyl acetate under a vapor of 2% glutaraldehyde. Grids will be visualized on a Zeiss EM 10 electron microscope at 80 kV. For Congo red staining, the incubated peptides will be placed onto gelatin-coated glass microscope slides and air-dried at 37° C. The slices will then be immersed in 0.2% Congo red dissolved in 80% aqueous ethanol saturated with NaCl for 60 min at room temperature, washed three times with water and visualized by polarized light microscopy.

### REFERENCES

Bauer, J. et al. Inflammation in the nervous system: the human perspective. 2001 Glia 36: 235-243.

Bien C G, Bauer J, Deckwerth T L, Wiendl H, Deckert M, Wiestler O D, Schramm J, Elger C E, Lassmann H. Destruction of neurons by cytotoxic T cells: a new pathogenic mechanism in Rasmussen's encephalitis. Ann Neurol 2002 March; 51(3):311-8

Blasko I, Marx F, Steiner E, Hartmann T, Grubeck-Loebenstein B TNFalpha plus IFNgamma induce the production of Alzheimer beta-amyloid peptides and decrease the secretion of APPs. FASEB J 1999 January; 13(1):63-8

Buscighio, J. et al. Generation of beta-amyloid in the secretory pathway in neuronal and normeuronal cells. (1993) Proc Natl Acad Sci USA 90:2092-2096

Buus S, Stryhn A, Winther K, Kirkby N, Pedersen L O Receptor-ligand interactions measured by an improved spun column chromatography technique. A high efficiency and high throughput size separation method. Biochim Biophys Acta. Apr. 13, 1995; 1243(3):453-60.

Check, E. Nerve inflammation halts trial for Alzheimer's drug. Nature. Jan. 31, 2002;415(6871):462.

Gay, F. W. et al. The application of multifactorial cluster analysis in the staging of plaques in early multiple sclerosis. Identification and characterization of the primary demyelinating lesion. Brain. 1997 August;120 (Pt 8):1461-83.

Geddes, J. W. et al 1999 N-terminus truncated beta-amyloid peptides and C-terminus truncated secreted forms of amyloid precursor protein: distinct roles in the pathogenesis of Alzheimer's disease. Neurobiol Aging. 1999 20(1):75-9

Glenner, G. G. and Wong, C. W. Alzheimer's disease: initial report of the purification and characterization of a novel cerebrovascular amyloid protein. (1984) Biochem Biophys Res Commun 120:885-890

Griffin D E, Levine B, Tyor W R, Irani D N. The immune response in viral encephalitis. Semin Immunol 1992 April; 4(2):111-9

Haass, C. et al. Amyloid beta-peptide is produced by cultured cells during normal metabolism. (1992) Nature 359:322-325

Haas C et al (1994) “Mutations associated with a locus for familial Alzheimer's disease result in alternative processing of amyloid b-protein precursor.” J Biol. Chem. 269, 17741-17748.

Higgins L S et al (1996) “p3 b amyloid peptide has a unique and potentially pathogenic immunohistochemical profile in Alzheimer's disease brain.” Am J. Pathol 149, 585-596.

Janus, C. et al. A beta peptide immunization reduces behavioural impairment and plaques in a model of Alzheimer's disease. 2000 Nature 408, 979-982.

Joachim, C. L. et al. Protein chemical and immunocytochemical studies of meningovascular beta-amyloid protein in Alzheimer's disease and normal aging. (1988) Brain Res 474:100-111

Kang, J. et al. The precursor of Alzheimer's disease amyloid A4 protein resembles a cell-surface receptor. Nature. Feb. 19-25, 1987; 325(6106):733-6

Kimura T, Griffin D E. The role of CD8(+) T cells and major histocompatibility complex class I expression in the central nervous system of mice infected with neurovirulent Sindbis virus. J Virol 2000 July; 74(13):6117-25

Lalowski M et al., The “nonamyloidogenic” p3 fragment (amyloid beta17-42) is a major constituent of Down's syndrome cerebellar preamyloid. 1996 J Biol Chem 271(52):33623-31

Larner A J (1999) “Hypothesis: amyloid b peptides truncated at the N-terminus contribute to the pathogenesis of Alzheimer's disease.” Neurbiol. Of Aging 20, 65-69.

Marx F, Blasko I, Zisterer K, Grubeck-Loebenstein B. Transfected human B cells: a new model to study the functional and immunostimulatory consequences of APP production. Exp Gerontol 1999 September; 34(6):783-95

Morgan, D, et al. A beta peptide vaccination prevents memory loss in an animal model of Alzheimer's disease. 2000 Nature 408, 982-985.

Mori, H. et al. Mass spectrometry of purified amyloid beta protein in Alzheimer's disease. (1992) J Biol Chem 267:17082-17806

Naslund, J. et al. Relative abundance of Alzheimer A beta amyloid peptide variants in Alzheimer disease and normal aging. (1994) Proc Natl Acad Sci USA 91:8378-8382

Neumann, H. et al. Major histocompatibility complex (MIC) class I gene expression in single neurons of the central nervous system: differential regulation by interferon (IFN)-gamma and tumor necrosis factor (TNF)-alpha. 1997 J Exp Med 185: 305-316.

Oldstone, M. B., Southern, P. J. Trafficking of activated cytotoxic T lymphocytes into the central nervous system: use of a transgenic model. 1993 J Neuroimuunol 46:25-31.

Ostergaard Pedersen L, et. al Efficient assembly of recombinant major histocompatibility complex class I molecules with preformed disulfide bonds. Eur J. Immunol. 2001 October; 31(10):2986-96

Parker, K. C., M. A. Bednarek, and J. E. Coligan. 1994. Scheme for ranking potential HLA-A2 binding peptides based on independent binding of individual peptide side-chains. J. Immunol. 152:163.

Pike C J et al (1995) Amino-terminal deletions enhance aggregation of P-amyloid peptides in vitro. J Biol Chem 270, 23895-8.

Prelli, F. et al. Differences between vascular and plaque core amyloid in Alzheimer's disease. (1988) J Neurochem 51:648-651

Rammensee, Hans-Georg, Jutta Bachmann, Niels Nikolaus Emmerich, Oskar Alexander Bachor, Stefan Stevanovic: SYFPEITHI: database for MHC ligands and peptide motifs. Immunogenetics (1999) 50: 213-219 (access via: http://www.uni-tuebingen.de/uni/kxi/)

Reche P A, Glutting J P and Reinherz E L. Prediction of MHC Class I Binding Peptides Using Profile Motifs. Human Immunology 63, 701 709 (2002).

Roher, A. E. et al. beta-Amyloid-(1-42) is a major component of cerebrovascular amyloid deposits: implications for the pathology of Alzheimer disease. (1993) Proc Natl Acad Sci USA 90:10836-10840

Schenk, D. et al. Immunization with amyloid-beta attenuates Alzheimer-disease-like pathology in the PDAPP mouse. 1999 Nature 400, 173-177.

Seubert, P. et al. Isolation and quantification of soluble Alzheimer's beta-peptide from biological fluids. (1992) Nature 359:325-327

Sturniolo. T., et al., Generation of tissue-specific and promiscuous HLA ligand database using DNA microarrays and virtual HLA class II matrices. Nat. Biotechnol. 17. 555-561(1999)

Tekirian T L Commentary: Abeta N-Terminal Isoforms: Critical contributors in the course of AD pathophysiology. J Alzheimers Dis. 2001 April; 3(2):241-248.

Traugott, U. Multiple sclerosis: relevance of class I and class II MHC-expressing cells to lesion development. 1987 J Neurimmunol 16: 283-302.

Vass, K. and Lassmann, H. Intrathecal application of interferon gamma. Progressive appearance of MHC antigens within the rat nervous system. 1990 Am J. Pathol 137: 789-800.

What is claimed is:

1. An isolated amyloid beta peptide or homologue thereof, selected according to the method comprising the steps of:
a. determining the binding value of each amino acid of a subsequence of amyloid beta peptide or homologue thereof upon binding to a HLA class 1 and/or class II molecule of interest;
b. determining the resulting score of all amino acids of the subsequence, based on the binding value of each amino acids obtained in step a; and
c. comparing said resulting score to a preselected value, wherein a subsequence with a resulting score, which is less than said preselected value is then selected as contained in the isolated amyloid beta peptide or homologue thereof.

2. The isolated amyloid beta peptide or homologue thereof of claim 1, wherein said peptide obtained in step C is further being assessed for lack of its ability to induce a T-cell response.

3. The isolated amyloid beta peptide or homologue thereof of claim 2, wherein lack of ability to induce a T-cell response is assessed as lack of ability to induce T-cell proliferation.

4. The isolated amyloid beta peptide or homologue thereof of claim 2, wherein lack of ability to induce a T-cell response is assessed as lack of ability to induce T- cell cytotoxicity.

5. The isolated amyloid beta peptide or homologue thereof of claim 2, wherein lack of ability to induce a T-cell response is assessed as lack of ability to induce cytokines.

6. The isolated amyloid beta peptide or homologue thereof of claim 2, wherein lack of ability to induce a T-cell response is assessed as lack of ability to detect T-cell activation markers.

7. The isolated amyloid beta peptide or homologue thereof of claim 2, wherein lack of ability to induce a T-cell response is assessed as lack of ability to detect specific T-cell receptors.

8. The isolated amyloid beta peptide or homologue thereof of claim 1, wherein said peptide for preparing a vaccine comprising amyloid beta or homologue thereof is further being assessed for lack of fibrillogenicity.

9. The isolated amyloid beta peptide or homologue thereof of claim 1, wherein said peptide for preparing a vaccine comprising amyloid beta or homologue thereof is further being assessed for lack of beta sheet structure.

10. The isolated amyloid beta peptide or homologue thereof of claim 1, wherein said peptide is further being assessed for lack of toxicity.

11. The isolated amyloid beta peptide or homologue thereof of claim 1, wherein said peptide is further being assessed for lack of cytotoxicity.

12. The isolated amyloid beta peptide or homologue thereof of claim 1, wherein said peptide is further being assessed for its ability to induce an antibody response.

13. A vacccine comprising the isolated amyloid beta peptide or homolog thereof of claim 1, whereby the amyloid beta peptide or homologue thereof lacks the ability to induce a T-cell response.

14. The vaccine of claim 13, wherein lack of ability to induce a T-cell response is assessed as lack of ability to induce T-cell proliferation.

15. The vaccine of claim 13, wherein lack of ability to induce a T-cell response is assessed as lack of ability to induce T-cell cytotoxicity.

16. The vaccine of claim 13, wherein lack of ability to induce a T-cell response is assessed as lack of ability to induce cytokines.

17. The vaccine of claim 13, wherein lack of ability to induce a T-cell response is assessed as lack of ability to detect T-cell activation markers.

18. The vaccine of claim 13, wherein lack of ability to induce a T-cell response is assessed as lack of ability to detect specific T-cell receptors.

19. A vaccine comprising an amyloid beta peptide or homologue thereof and a carrier or a diluent, wherein the peptide or homologue thereof are selected according to the method comprising the steps of:
a. determining the binding value of each amino acid of a subsequence of amyloid beta peptide or homologue thereof for binding to a HLA class 1 and/or class II molecule of interest;
b. determining the resulting score of all amino acids of the subsequence based on the binding value of each amino acid obtained in step a; and
C. comparing said resulting score to a preselected value, wherein a subsequence with a resulting score, which is less than said preselected value is then selected as contained in the isolated amyloid beta peptide or homologue thereof of the vaccine.

20. The vaccine of claim 19, wherein said peptide obtained in step C is further being assessed for lack of its ability to induce a T-cell response.

21. The vaccine of claim 19, wherein lack of ability to induce a T-cell response is assessed as lack of ability to induce T-cell proliferation.

22. The vaccine of claim 19, wherein lack of ability to induce a T-cell response is assessed as lack of ability to induce T- cell cytotoxicity.

23. The vaccine of claim 19, wherein lack of ability to induce a T-cell response is assessed as lack of ability to induce cytokines.

24. The vaccine of claim 19, wherein lack of ability to induce a T-cell response is assessed as lack of ability to detect T-cell activation markers.

25. The vaccine of claim 19, wherein lack of ability to induce a T-cell response is assessed as lack of ability to detect specific T-cell receptors.

26. The vaccine of claim 19, wherein said peptide for preparing a vaccine comprising amyloid beta or homologue thereof is further being assessed for lack of fibrillogenicity.

27. The vaccine of claim 19, wherein said peptide for preparing a vaccine comprising amyloid beta or homologue thereof is further being assessed for lack of beta sheet structure.

28. The vaccine of claim 19, wherein said peptide for preparing a vaccine comprising amyloid beta or homologous thereof is further being assessed for lack of toxicity.

29. The vaccine of claim 19, wherein said peptide for preparing a vaccine comprising amyloid beta or homologous thereof is further being assessed for lack of cytotoxicity.

30. The vaccine of claim 19, wherein said peptide for preparing a vaccine comprising amyloid beta or homologue thereof is further being assessed for its ability to induce an antibody response.

31. A vaccine comprising an amyloid beta peptide or homologue thereof, whereby the amyloid beta peptide or homologue thereof lacks the ability to induce a T-cell response.

32. The vaccine of claim 31, wherein lack of ability to induce a T-cell response is assessed as lack of ability to induce T-cell proliferation.

33. The vaccine of claim 31, wherein lack of ability to induce a T-cell response is assessed as lack of ability to induce T-cell cytotoxicity.

34. The vaccine of claim 31, wherein lack of ability to induce a T-cell response is assessed as lack of ability to induce cytokines.

35. The vaccine of claim 31, wherein lack of ability to induce a T-cell response is assessed as lack of ability to detect T-cell activation markers.

36. The vaccine of claim 31, wherein lack of ability to induce a T-cell response is assessed as lack of ability to detect specific T-cell receptors.

37. A method of determining T-cell epitopes within amyloid beta peptide or homologue thereof comprising the steps of:
a. determining the binding value of each amino acid of a subsequence of amyloid beta peptide or homologue thereof upon binding to a HLA class 1 and/or class II molecule of interest;
b. determining the -resulting score of all amino acids of the subsequence based on the binding value of each amino acids obtained in step a; and
C. comparing said resulting score to a preselected value, to predict presence of T-cell epitopes within amyloid beta peptide or homologue thereof.

38. A method of predicting the reaction of an individual to a vaccine, which comprises amyloid beta peptide or homologue thereof, comprising the following steps:
a. obtaining a sample from a subject;
determining the HLA haplotype of said subject;
C. determining the binding value of each amino acid of a subsequence of amyloid beta peptide or homologue thereof to HLA molecules of said individual;
d. determining the resulting score of all amino acid of the subsequence based on the binding value of each amino acids obtained in step c; and;
e. comparing said resulting score to a preselected value, wherein if said resulting score is higher than said preselected score, the individual has the potential to develop T-cell responses immune response, and if said resulting score is lower than said preselected score the individual does not have the potential of developing a T-cell responses.

39. The method of claim 38, wherein said sample comprises body fluid or tissue.

40. The method of claim 38, wherein said body fluid comprises cerebral spinal fluid or blood.

41. The method of claim 38, wherein the tissue comprises skin or nose epithelium.

42. A method of matching a vaccine comprising a beta amyloid or homologue peptide thereof to an individual, for immunization of an individual wherein the based on the HLA haplotype of the individual comprising:
a. obtaining a sample from a subject;
determining the HLA haplotype of said subject;
c. determining the binding value of each amino acid of a subsequence of amyloid beta peptide or homologue thereof to HLA molecules of said individual;
d. determining the resulting score of all amino acid of the subsequence based on the binding value of each amino acids obtained in step a; and
comparing said resulting score to a preselected value, wherein if said resulting score is lower than said preselected score, the beta amyloid or homologue thereof is selected for preparing a vaccine comprising beta amyloid peptide or homologous thereof for immunization of an individual based on the haplotype of the individual and if said resulting score is higher than said preselected score, the beta amyloid or homologue thereof is not selected for immunization of the individual based on the haplotype of the individual.

43. The method of claim 42, wherein said sample comprises body fluid or tissue.

44. The method of claim 42, wherein said body fluid comprises cerebral spinal fluid or blood.

45. The method of claim 42, wherein the tissue comprises skin or nose epithelium.

46. The method of claim 42, wherein said peptide obtained in step e is further being assessed for lack of its ability to induce T-cell responses.

47. The method of claim 46, wherein lack of ability to induce a T-cell response is assessed as lack of ability to induce T-cell proliferation.

48. The method of claim 46, wherein lack of ability to induce a T-cell response is assessed as lack of ability to induce T- cell cytotoxicity.

49. The method of claim 46, wherein lack of ability to induce a T-cell response is assessed as lack of ability to induce cytokines.

50. The method of claim 46, wherein lack of ability to induce a T-cell response is assessed as lack of ability to detect T-cell activation markers.

51. The method of claim 46, wherein lack of ability to induce a T-cell response is assessed as lack of ability to detect specific T-cell receptors.

52. The method of claim 42, wherein said peptide for preparing a vaccine comprising amyloid beta or homologue thereof is further being assessed for lack of fibrillogenicity.

53. The method of claim 42, wherein said peptide for preparing a vaccine comprising amyloid beta or homologue thereof is further being assessed for lack of beta sheet structure.

54. The method of claim 42, wherein said peptide for preparing a vaccine comprising amyloid beta or homologous thereof is further being assessed for lack of toxicity.

55. The method of claim 42, wherein said peptide for preparing a vaccine comprising amyloid beta or homologous thereof is further being assessed for lack of cytotoxicity.

56. The method of claim 42, wherein said peptide for preparing a vaccine comprising amyloid beta or homologoue thereof is further being assessed for its ability to induce antibody responses.

57. A kit for matching a vaccine comprising amyloid beta peptide or homologue thereof to an individual based on the HLA haplotype of the individual comprising:
a) a means for obtaining a blood sample from the individual;
b) a means for determining the HLA haplotype of the individual; and
c) a means for determination of the binding of subsequence of amyloid beta or homologous to HLA haplotype of the individual.

58. A method of preventing the formation or progression of amyloid plaques using the vaccine of claims 13.

59. A method of preventing the formation or progression of amyloid plaques using the amyloid beta peptide or homologue thereof of claim 1.


SEQUENCE LISTING (187 sequences)

SEQ ID NO: 1 - protein, 10 aa, artificial sequence
  algorithm generated
FLPSDYFPSV

SEQ ID NO: 2 - protein, 43 aa, artificial sequence
  algorithm generated
DAEFRHDSGYEVHHQKLVFFAEDVGSNKGAIIGLMVGGVVIAT

SEQ ID NO: 3 - protein, 9 aa, artificial sequence
  algorithm generated
KLVFFAEDV

SEQ ID NO: 4 - protein, 9 aa, artificial sequence
  algorithm generated
GLMVGGVVI

SEQ ID NO: 5 - protein, 9 aa, artificial sequence
  algorithm generated
YEVHHQKLV

SEQ ID NO: 6 - protein, 9 aa, artificial sequence
  algorithm generated
LMVGGVVIA

SEQ ID NO: 7 - protein, 9 aa, artificial sequence
  algorithm generated
IIGLMVGGV

SEQ ID NO: 8 - protein, 9 aa, artificial sequence
  algorithm generated
MVGGVVIAT

SEQ ID NO: 9 - protein, 9 aa, artificial sequence
  algorithm generated
KGAIIGLMV

SEQ ID NO: 10 - protein, 9 aa, artificial sequence
  algorithm generated
FRHDSGYEV

SEQ ID NO: 11 - protein, 9 aa, artificial sequence
  algorithm generated
IGLMVGGVV

SEQ ID NO: 12 - protein, 9 aa, artificial sequence
  algorithm generated
VGSNKGAII

SEQ ID NO: 13 - protein, 30 aa, artificial sequence
  algorithm generated
DAEFRHDSGYEVHHQKLEEFAEDVGSNKGA

SEQ ID NO: 14 - protein, 9 aa, artificial sequence
  algorithm generated
KLEEFAEDV

SEQ ID NO: 15 - protein, 9 aa, artificial sequence
  algorithm generated
FRHDSGYEV

SEQ ID NO: 16 - protein, 9 aa, artificial sequence
  algorithm generated
AEFRHDSGY

SEQ ID NO: 17 - protein, 9 aa, artificial sequence
  algorithm generated
YEVHHQKLE

SEQ ID NO: 18 - protein, 9 aa, artificial sequence
  algorithm generated
SGYEVHHQK

SEQ ID NO: 19 - protein, 9 aa, artificial sequence
  algorithm generated
QKLEEFAED

SEQ ID NO: 20 - protein, 9 aa, artificial sequence
  algorithm generated
AEDVGSNKG

SEQ ID NO: 21 - protein, 9 aa, artificial sequence
  algorithm generated
EDVGSNKGA

SEQ ID NO: 22 - protein, 9 aa, artificial sequence
  algorithm generated
EEFAEDVGS

SEQ ID NO: 23 - protein, 9 aa, artificial sequence
  algorithm generated
HHQKLEEFA

SEQ ID NO: 24 - protein, 30 aa, artificial sequence
  algorithm generated
DAEFRHDSGYEVHHQKLVFFAEDVGSNKGA

SEQ ID NO: 25 - protein, 9 aa, artificial sequence
  algorithm generated
KLVFFAEDV

SEQ ID NO: 26 - protein, 9 aa, artificial sequence
  algorithm generated
YEVHHQKLV

SEQ ID NO: 27 - protein, 9 aa, artificial sequence
  algorithm generated
FRHDSGYEV

SEQ ID NO: 28 - protein, 9 aa, artificial sequence
  algorithm generated
HHQKLVFFA

SEQ ID NO: 29 - protein, 9 aa, artificial sequence
  algorithm generated
LVFFAEDVG

SEQ ID NO: 30 - protein, 30 aa, artificial sequence
  algorithm generated
DAEFRHDSGYEVHHQKEEFFAEDVGSNKGA

SEQ ID NO: 31 - protein, 9 aa, artificial sequence
  algorithm generated
KEEFFAEDV

SEQ ID NO: 32 - protein, 9 aa, artificial sequence
  algorithm generated
FRHDSGYEV

SEQ ID NO: 33 - protein, 9 aa, artificial sequence
  algorithm generated
AEFRHDSGY

SEQ ID NO: 34 - protein, 9 aa, artificial sequence
  algorithm generated
HHQKEEFFA

SEQ ID NO: 35 - protein, 9 aa, artificial sequence
  algorithm generated
YEVHHQKEE

SEQ ID NO: 36 - protein, 30 aa, artificial sequence
  algorithm generated
DAEFRHDSGYEVHHQKDDFFAEDVGSNKGA

SEQ ID NO: 37 - protein, 9 aa, artificial sequence
  algorithm generated
KDDFFAEDV

SEQ ID NO: 38 - protein, 9 aa, artificial sequence
  algorithm generated
FRHDSGYEV

SEQ ID NO: 39 - protein, 9 aa, artificial sequence
  algorithm generated
AEFRHDSGY

SEQ ID NO: 40 - protein, 9 aa, artificial sequence
  algorithm generated
HHQKDDFFA

SEQ ID NO: 41 - protein, 9 aa, artificial sequence
  algorithm generated
YEVHHQKDD

SEQ ID NO: 42 - protein, 30 aa, artificial sequence
  algorithm generated
DAEFRHDSGYEVHHQKKKFFAEDVGSNKGA

SEQ ID NO: 43 - protein, 9 aa, artificial sequence
  algorithm generated
KKKFFAEDV

SEQ ID NO: 44 - protein, 9 aa, artificial sequence
  algorithm generated
AEFRHDSGY

SEQ ID NO: 45 - protein, 9 aa, artificial sequence
  algorithm generated
HHQKKKFFA

SEQ ID NO: 46 - protein, 9 aa, artificial sequence
  algorithm generated
YEVHHQKKK

SEQ ID NO: 47 - protein, 9 aa, artificial sequence
  algorithm generated
YEVHHQKKK

SEQ ID NO: 48 - protein, 9 aa, artificial sequence
  algorithm generated
FRHDSGYEV

SEQ ID NO: 49 - protein, 9 aa, artificial sequence
  algorithm generated
AEFRHDSGY

SEQ ID NO: 50 - protein, 9 aa, artificial sequence
  algorithm generated
YEVHHQKEE

SEQ ID NO: 51 - protein, 9 aa, artificial sequence
  algorithm generated
SGYEVHHQK

SEQ ID NO: 52 - protein, 9 aa, artificial sequence
  algorithm generated
KEDDFAEDV

SEQ ID NO: 53 - protein, 9 aa, artificial sequence
  algorithm generated
FRHDSGYEV

SEQ ID NO: 54 - protein, 9 aa, artificial sequence
  algorithm generated
AEFRHDSGY

SEQ ID NO: 55 - protein, 9 aa, artificial sequence
  algorithm generated
YEVHHQKED

SEQ ID NO: 56 - protein, 9 aa, artificial sequence
  algorithm generated
SGYEVHHQK

SEQ ID NO: 57 - protein, 9 aa, artificial sequence
  algorithm generated
KLVFFAEDV

SEQ ID NO: 58 - protein, 9 aa, artificial sequence
  algorithm generated
GLMVGGVVI

SEQ ID NO: 59 - protein, 9 aa, artificial sequence
  algorithm generated
YEVHHQKLV

SEQ ID NO: 60 - protein, 9 aa, artificial sequence
  algorithm generated
IIGLMVGGV

SEQ ID NO: 61 - protein, 9 aa, artificial sequence
  algorithm generated
KEEFFAEDV

SEQ ID NO: 62 - protein, 9 aa, artificial sequence
  algorithm generated
FRHDSGYEV

SEQ ID NO: 63 - protein, 9 aa, artificial sequence
  algorithm generated
AEFRHDSGY

SEQ ID NO: 64 - protein, 9 aa, artificial sequence
  algorithm generated
HHQKEEFFA

SEQ ID NO: 65 - protein, 9 aa, artificial sequence
  algorithm generated
YEVHHQKEE

SEQ ID NO: 66 - protein, 36 aa, artificial sequence
  algorithm generated
KKKKKKDAEFRHDSGYEVHHQKEEFFAEDVGSNKGA

SEQ ID NO: 67 - protein, 9 aa, artificial sequence
  algorithm generated
FAEDVGSNK

SEQ ID NO: 68 - protein, 9 aa, artificial sequence
  algorithm generated
DAEFRHDSG

SEQ ID NO: 69 - protein, 9 aa, artificial sequence
  algorithm generated
EVHHQKEEF

SEQ ID NO: 70 - protein, 9 aa, artificial sequence
  algorithm generated
SGYEVHHQK

SEQ ID NO: 71 - protein, 9 aa, artificial sequence
  algorithm generated
KEEFFAEDV

SEQ ID NO: 72 - protein, 9 aa, artificial sequence
  algorithm generated
KEEFFAEDV

SEQ ID NO: 73 - protein, 9 aa, artificial sequence
  algorithm generated
FRHDSGYEV

SEQ ID NO: 74 - protein, 9 aa, artificial sequence
  algorithm generated
KDAEFRHDS

SEQ ID NO: 75 - protein, 9 aa, artificial sequence
  algorithm generated
AEFRHDSGY

SEQ ID NO: 76 - protein, 9 aa, artificial sequence
  algorithm generated
HHQKEEFFA

SEQ ID NO: 77 - protein, 9 aa, artificial sequence
  algorithm generated
KEEFFAEDV

SEQ ID NO: 78 - protein, 9 aa, artificial sequence
  algorithm generated
FRHDSGYEV

SEQ ID NO: 79 - protein, 9 aa, artificial sequence
  algorithm generated
AEFRHDSGY

SEQ ID NO: 80 - protein, 9 aa, artificial sequence
  algorithm generated
HHQKEEFFA

SEQ ID NO: 81 - protein, 9 aa, artificial sequence
  algorithm generated
KDAEFRHDS

SEQ ID NO: 82 - protein, 9 aa, artificial sequence
  algorithm generated
FAEDVGSNK

SEQ ID NO: 83 - protein, 9 aa, artificial sequence
  algorithm generated
SGYEVHHQK

SEQ ID NO: 84 - protein, 9 aa, artificial sequence
  algorithm generated
EVHHQKEEF

SEQ ID NO: 85 - protein, 9 aa, artificial sequence
  algorithm generated
AEFRHDSGY

SEQ ID NO: 86 - protein, 9 aa, artificial sequence
  algorithm generated
KKKKDAEFR

SEQ ID NO: 87 - protein, 9 aa, artificial sequence
  algorithm generated
EVHHQKEEF

SEQ ID NO: 88 - protein, 9 aa, artificial sequence
  algorithm generated
GYEVHHQKE

SEQ ID NO: 89 - protein, 9 aa, artificial sequence
  algorithm generated
FFAEDVGSN

SEQ ID NO: 90 - protein, 9 aa, artificial sequence
  algorithm generated
EFFAEDVGS

SEQ ID NO: 91 - protein, 9 aa, artificial sequence
  algorithm generated
KKKKKDAEF

SEQ ID NO: 92 - protein, 36 aa, artificial sequence; identical to SEQ ID NO: 66
  algorithm generated

SEQ ID NO: 93 - protein, 9 aa, artificial sequence
  algorithm generated
KEEFFAEDV

SEQ ID NO: 94 - protein, 9 aa, artificial sequence
  algorithm generated
EEFFAEDVG

SEQ ID NO: 95 - protein, 9 aa, artificial sequence
  algorithm generated
EVHHQKEEF

SEQ ID NO: 96 - protein, 9 aa, artificial sequence
  algorithm generated
SGYEVHHQK

SEQ ID NO: 97 - protein, 9 aa, artificial sequence
  algorithm generated
AEFRHDSGY

SEQ ID NO: 98 - protein, 9 aa, artificial sequence
  algorithm generated
KEEFFAEDV

SEQ ID NO: 99 - protein, 9 aa, artificial sequence
  algorithm generated
EDVGSNKGA

SEQ ID NO: 100 - protein, 9 aa, artificial sequence
  algorithm generated
AEFRHDSGY

SEQ ID NO: 101 - protein, 9 aa, artificial sequence
  algorithm generated
EEFFAEDVG

SEQ ID NO: 102 - protein, 9 aa, artificial sequence
  algorithm generated
YEVHHQKEE

SEQ ID NO: 103 - protein, 9 aa, artificial sequence
  algorithm generated
DAEFRHDSG

SEQ ID NO: 104 - protein, 9 aa, artificial sequence
  algorithm generated
FAEDVGSNK

SEQ ID NO: 105 - protein, 9 aa, artificial sequence
  algorithm generated
FRHDSGYEV

SEQ ID NO: 106 - protein, 9 aa, artificial sequence
  algorithm generated
SGYEVHHQK

SEQ ID NO: 107 - protein, 9 aa, artificial sequence
  algorithm generated
KEEFFAEDV

SEQ ID NO: 108 - protein, 9 aa, artificial sequence
  algorithm generated
SGYEVHHQK

SEQ ID NO: 109 - protein, 9 aa, artificial sequence
  algorithm generated
FRHDSGYEV

SEQ ID NO: 110 - protein, 9 aa, artificial sequence
  algorithm generated
FAEDVGSNK

SEQ ID NO: 111 - protein, 9 aa, artificial sequence
  algorithm generated
EFFAEDVGS

SEQ ID NO: 112 - protein, 9 aa, artificial sequence
  algorithm generated
DAEFRHDSG

SEQ ID NO: 113 - protein, 9 aa, artificial sequence
  algorithm generated
SGYEVHHQK

SEQ ID NO: 114 - protein, 9 aa, artificial sequence
  algorithm generated
DAEFRHDSG

SEQ ID NO: 115 - protein, 9 aa, artificial sequence
  algorithm generated
FAEDVGSNK

SEQ ID NO: 116 - protein, 9 aa, artificial sequence
  algorithm generated
FRHDSGYEV

SEQ ID NO: 117 - protein, 9 aa, artificial sequence
  algorithm generated
KEEFFAEDV

SEQ ID NO: 118 - protein, 9 aa, artificial sequence
  algorithm generated
HQKEEFFAE

SEQ ID NO: 119 - protein, 9 aa, artificial sequence
  algorithm generated
EVHHQKEEF

SEQ ID NO: 120 - protein, 9 aa, artificial sequence
  algorithm generated
KKKKKDAEF

SEQ ID NO: 121 - protein, 9 aa, artificial sequence
  algorithm generated
VHHQKEEFF

SEQ ID NO: 122 - protein, 9 aa, artificial sequence
  algorithm generated
AEFRHDSGY

SEQ ID NO: 123 - protein, 9 aa, artificial sequence
  algorithm generated
EVHHQKEEF

SEQ ID NO: 124 - protein, 9 aa, artificial sequence
  algorithm generated
KKKKKDAEF

SEQ ID NO: 125 - protein, 9 aa, artificial sequence
  algorithm generated
AEFRHDSGY

SEQ ID NO: 126 - protein, 9 aa, artificial sequence
  algorithm generated
VHHQKEEFF

SEQ ID NO: 127 - protein, 9 aa, artificial sequence
  algorithm generated
HQKEEFFAE

SEQ ID NO: 128 - protein, 9 aa, artificial sequence
  algorithm generated
YEVHHQKLV

SEQ ID NO: 129 - protein, 9 aa, artificial sequence
  algorithm generated
FRHDSGYEV

SEQ ID NO: 130 - protein, 9 aa, artificial sequence
  algorithm generated
FAEDVGSNK

SEQ ID NO: 131 - protein, 9 aa, artificial sequence
  algorithm generated
VGSNKGAII

SEQ ID NO: 132 - protein, 9 aa, artificial sequence
  algorithm generated
IGLMVGGVV

SEQ ID NO: 133 - protein, 9 aa, artificial sequence
  algorithm generated
VFFAEDVGS

SEQ ID NO: 134 - protein, 9 aa, artificial sequence
  algorithm generated
LVFFAEDVG

SEQ ID NO: 135 - protein, 9 aa, artificial sequence
  algorithm generated
VHHQKLVFF

SEQ ID NO: 136 - protein, 9 aa, artificial sequence
  algorithm generated
AEFRHDSGY

SEQ ID NO: 137 - protein, 9 aa, artificial sequence
  algorithm generated
FAEDVGSNK

SEQ ID NO: 138 - protein, 9 aa, artificial sequence
  algorithm generated
YEVHHQKLV

SEQ ID NO: 139 - protein, 9 aa, artificial sequence
  algorithm generated
FAEDVGSNK

SEQ ID NO: 140 - protein, 9 aa, artificial sequence
  algorithm generated
FFAEDVGSN

SEQ ID NO: 141 - protein, 9 aa, artificial sequence
  algorithm generated
FRHDSGYEV

SEQ ID NO: 142 - protein, 9 aa, artificial sequence
  algorithm generated
VGSNKGAII

SEQ ID NO: 143 - protein, 9 aa, artificial sequence
  algorithm generated
FAEDVGSNK

SEQ ID NO: 144 - protein, 9 aa, artificial sequence
  algorithm generated
IGLMVGGVV

SEQ ID NO: 145 - protein, 9 aa, artificial sequence
  algorithm generated
YEVHHQKLV

SEQ ID NO: 146 - protein, 9 aa, artificial sequence
  algorithm generated
FRHDSGYEV

SEQ ID NO: 147 - protein, 9 aa, artificial sequence
  algorithm generated
SGYEVHHQK

SEQ ID NO: 148 - protein, 9 aa, artificial sequence
  algorithm generated
FAEDVGSNK

SEQ ID NO: 149 - protein, 9 aa, artificial sequence
  algorithm generated
FRHDSGYEV

SEQ ID NO: 150 - protein, 9 aa, artificial sequence
  algorithm generated
YEVHHQKLV

SEQ ID NO: 151 - protein, 9 aa, artificial sequence
  algorithm generated
FFAEDVGSN

SEQ ID NO: 152 - protein, 9 aa, artificial sequence
  algorithm generated
LMVGGVVIA

SEQ ID NO: 153 - protein, 9 aa, artificial sequence
  algorithm generated
EFRHDSGYE

SEQ ID NO: 154 - protein, 9 aa, artificial sequence
  algorithm generated
RHDSGYEVH

SEQ ID NO: 155 - protein, 9 aa, artificial sequence
  algorithm generated
DVGSNKGAI

SEQ ID NO: 156 - protein, 9 aa, artificial sequence
  algorithm generated
EVHHQKLVF

SEQ ID NO: 157 - protein, 9 aa, artificial sequence
  algorithm generated
KLVFFAEDV

SEQ ID NO: 158 - protein, 9 aa, artificial sequence
  algorithm generated
FRHDSGYEV

SEQ ID NO: 159 - protein, 9 aa, artificial sequence
  algorithm generated
HQKLEEFAE

SEQ ID NO: 160 - protein, 9 aa, artificial sequence
  algorithm generated
FAEDVGSNK

SEQ ID NO: 161 - protein, 9 aa, artificial sequence
  algorithm generated
YEVHHQKLE

SEQ ID NO: 162 - protein, 9 aa, artificial sequence
  algorithm generated
LEEFAEDVG

SEQ ID NO: 163 - protein, 9 aa, artificial sequence
  algorithm generated
EEFAEDVGS

SEQ ID NO: 164 - protein, 9 aa, artificial sequence
  algorithm generated
LEEFAEDVG

SEQ ID NO: 165 - protein, 9 aa, artificial sequence
  algorithm generated
VHHQKLEEF

SEQ ID NO: 166 - protein, 9 aa, artificial sequence
  algorithm generated
AEFRHDSGY

SEQ ID NO: 167 - protein, 9 aa, artificial sequence
  algorithm generated
HHQKLEEFA

SEQ ID NO: 168 - protein, 9 aa, artificial sequence
  algorithm generated
YEVHHQKLE

SEQ ID NO: 169 - protein, 9 aa, artificial sequence
  algorithm generated
HQKLEEFAE

SEQ ID NO: 170 - protein, 9 aa, artificial sequence
  algorithm generated
FAEDVGSNK

SEQ ID NO: 171 - protein, 9 aa, artificial sequence
  algorithm generated
KKKKDAEFR

SEQ ID NO: 172 - protein, 9 aa, artificial sequence
  algorithm generated
FRHDSGYEV

SEQ ID NO: 173 - protein, 9 aa, artificial sequence
  algorithm generated
FAEDVGSNK

SEQ ID NO: 174 - protein, 9 aa, artificial sequence
  algorithm generated
KKKDAEFRH

SEQ ID NO: 175 - protein, 9 aa, artificial sequence
  algorithm generated
FRHDSGYEV

SEQ ID NO: 176 - protein, 9 aa, artificial sequence
  algorithm generated
YEVHHQKLE

SEQ ID NO: 177 - protein, 9 aa, artificial sequence
  algorithm generated
QKLEEFAED

SEQ ID NO: 178 - protein, 9 aa, artificial sequence
  algorithm generated
FAEDVGSNK

SEQ ID NO: 179 - protein, 9 aa, artificial sequence
  algorithm generated
FRHDSGYEV

SEQ ID NO: 180 - protein, 9 aa, artificial sequence
  algorithm generated
YEVHHQKLE

SEQ ID NO: 181 - protein, 9 aa, artificial sequence
  algorithm generated
HQKLEEFAE

SEQ ID NO: 182 - protein, 9 aa, artificial sequence
  algorithm generated
LEEFAEDVG

SEQ ID NO: 183 - protein, 9 aa, artificial sequence
  algorithm generated
KKKKKKDAE

SEQ ID NO: 184 - protein, 9 aa, artificial sequence
  algorithm generated
KKKKDAEFR

SEQ ID NO: 185 - protein, 9 aa, artificial sequence
  algorithm generated
KLEEFAEDV

SEQ ID NO: 186 - protein, 9 aa, artificial sequence
  algorithm generated
HHQKLEEFA

SEQ ID NO: 187 - protein, 9 aa, artificial sequence
  algorithm generated
KKKKKDAEF